(19) United States
(12) Patent Application Publication
(10) Pub. No.: US 2013/0005610 A1
(43) Pub. Date: Jan. 3, 2013

(54) TOXICITY-ASSOCIATED GENES, GENETIC VARIANTS, AND USE THEREOF

(75) Inventor: Susanne Wagner, Salt Lake City, UT (US)

(73) Assignee: Myriad Genetics, Incorporated, Salt Lake City, UT (US)

(21) Appl. No.: 13/502,890

(22) PCT Filed: Oct. 19, 2010
(86) PCT No.: PCT/US10/53212

§ 371 Date: Sep. 4, 2012

Related U.S. Application Data
(60) Provisional application No. 61/252,899, filed on Oct. 19, 2009.

(51) Int. Cl. C12N 9/02; C07K 16/40; C40B 40/06; C07H 21/04; C12Q 1/68

(52) U.S. Cl. 506/16; 536/23.2; 435/189; 435/6.12; 536/24.31; 530/387.9

(57) ABSTRACT

Genes and genetic variants in human genomes are disclosed which are useful, inter alia, as diagnostic biomarkers.

### CROSS-REFERENCE TO RELATED APPLICATIONS

This application claims priority under 35 U.S.C. §119(e) to U.S. provisional application Ser. No. 61/252,899, filed Oct. 19, 2009, which is hereby incorporated by reference in its entirety.

### FIELD OF THE INVENTION

This invention generally relates to molecular genetics, particularly to the identification of variants associated with drug response.

### SEQUENCE LISTING

A formal Sequence Listing in computer readable form has been submitted electronically with this application as a text file. This text file, which is named “3017-02-1P-2009-10-16-SEQ-LIST-TXT-BGJ_ST25.txt”, was created on Oct. 16, 2009, and is 87,913 bytes in size. Its contents are incorporated by reference herein in their entirety.

### BACKGROUND OF THE INVENTION

Genetic polymorphic variations such as simple nucleotide polymorphisms (SNPs) are valuable tools for deciphering mechanisms of biological functions and understanding the underlying basis of human diseases. For example, SNPs in the Apolipoprotein E gene correlate with the risk of Alzheimer's disease. See U.S. Pat. No. 5,773,220.

Genetic polymorphic variations are also associated with varying response to drugs and natural environmental agents. For example, genetic variants in the drug-metabolizing enzyme thiopurine methyltransferase correlate with adverse drug reactions. See Krynetski et al., PHARM. RES., 16:342-349 (1999).

Merely finding a SNP in a gene may not be enough, however, since it may be difficult to determine whether a particular SNP will have any clinically significant phenotype based simply on its location in the gene. Thus, there is need in the art to both identify additional SNPs and determine their clinical significance, particularly those that may be associated with drug response and/or toxicity.

### SUMMARY OF THE INVENTION

The present invention is based on the discovery of several novel genetic variations in the DPYD gene and their association with particular responses to treatment with thymidylate synthase (TYMS) inhibitors (e.g., 5-fluorouracil). The variants are characterized in Table 1 below. Specifically, these variants are expected to be predictive of efficacy and/or toxicity in treatment with TYMS-inhibitors. Thus, the variants are useful in determining whether a patient will have toxicity to TYMS-inhibitors.

Accordingly, one aspect of the invention provides isolated nucleic acids comprising at least one of the variants listed in Table 1. Some embodiments provide an isolated human gene containing one or more of these variants. Some embodiments provide isolated nucleic acids whose nucleotide sequences comprise at least some specific number of contiguous nucleotides of the sequence of a variant of DPYD of the invention (e.g., SEQ ID NO:1), wherein the contiguous span encompasses and contains at least one of the variants listed in Table 1. Still other embodiments provide an isolated nucleic acid whose nucleotide sequence comprises a contiguous span of nucleotides of the sequence of at least one of SEQ ID NOs 63-97, wherein the contiguous span encompasses and contains at least one of the variants listed in Table 1.

In some embodiments the isolated nucleic acid of the invention comprises a variant listed in Table 1 at a particular position along its length. In some of these embodiments the variant is within some specific number of nucleotide positions of the center of said isolated nucleic acid. In some embodiments the variant is within some specific number of nucleotide positions of the 3′ end of said isolated nucleic acid. In some embodiments the variant is within some specific number of nucleotide positions of the 5′ end of said isolated nucleic acid.

In some embodiments the isolated nucleic acid of the invention hybridizes to or, together with one or more additional nucleic acid primers, amplifies only a nucleic acid comprising at least one variant listed in Table 1. In some of these embodiments the isolated nucleic acid (e.g., an oligonucleotide) hybridizes under stringent conditions (e.g., high stringency conditions) to a nucleic acid comprising a variant listed in Table 1 but not to a nucleic acid whose nucleotide sequence consists of the sequence of SEQ ID NO:100. In some embodiments the isolated nucleic acid, together with another primer, amplifies, under standard conditions and with standard reagents, a nucleic acid comprising a variant listed in Table 1 but not a nucleic acid whose nucleotide sequence comprises a portion of the sequence of SEQ ID NO:100.

Another aspect of the invention provides isolated polypeptides harboring one or more of the variants listed in Table 1. In some embodiments, polypeptides of the invention comprise at least some specific number of contiguous amino acids of a DPD variant of the invention (e.g., SEQ ID NO:2), wherein the contiguous span encompasses and contains at least one of the amino acid variants listed in Table 1.

Another aspect of the invention provides antibodies that bind immunologically to a polypeptide or peptide variant of the invention. Such antibodies may be generated based on the present novel sequence disclosures and various techniques known to those skilled in the art. The invention also provides hybridoma cell lines secreting antibodies of the invention.

Another aspect the invention provides diagnostic methods based on the variants (both nucleotide and amino acid) listed in Table 1. Generally, this aspect comprises determining whether a DPYD gene in a patient harbors a variant listed in Table 1. Determining whether a gene harbors a variant may involve testing the gene directly or testing it indirectly by assaying its expression products (e.g., mRNA, protein). Thus, in some embodiments the method comprises determining whether a genomic DPYD nucleic acid in a patient harbors a nucleotide variant listed in Table 1. In other embodiments the method comprises determining whether a patient's DPYD mRNA (or a cDNA encoded thereby) harbors a nucleotide variant listed in Table 1. In yet other embodiments the method comprises determining whether a DPD protein in a patient harbors an amino acid variant listed in Table 1.

In some embodiments the invention provides a method for determining whether a patient has an increased likelihood of toxicity to treatment comprising a TYMS-inhibitor, the method comprising determining whether a DPYD gene in a sample obtained from the patient harbors at least one variant listed in Table 1, wherein the patient has an increased likelihood of toxicity to treatment comprising a TYMS-inhibitor if the DPYD gene harbors a variant listed in Table 1. In some embodiments the method further comprises determining whether the patient has any additional markers relevant to response or toxicity to treatment with TYMS-inhibitors. In some of these embodiments at least one of these additional markers is chosen from Table 2.

Yet another aspect of the invention provides treatment methods based at least in part on whether a patient harbors a variant listed in Table 1. This aspect generally provides a treatment method comprising:
(1) determining whether a DPYD gene in a sample obtained from a patient harbors at least one variant listed in Table 1; and
(2) administering, prescribing or recommending a specific treatment regimen based at least in part on whether the patient harbors a variant listed in Table 1.

In some embodiments the specific treatment regimen comprises: administering, prescribing or recommending a treatment that does not comprise a TYMS-inhibitor; adjusting the initial dose of a TYMS-inhibitor; and/or monitoring said patient for toxicity to treatment comprising a TYMS-inhibitor. Those skilled in the art are, based on the present disclosure, capable of performing any combination of these. Thus, the invention provides treatment methods comprising administering, prescribing or recommending one or more of these based at least in part on whether the patient harbors a variant listed in Table 1.

In some embodiments the invention provides a treatment method further comprising determining whether a patient sample harbors any additional marker predictive of response, toxicity, or the absence of either in treatment comprising a TYMS-inhibitor. In these embodiments, monitoring the patient for toxicity, prescribing a treatment that does not comprise a TYMS-inhibitor, and/or adjusting the initial dose of a TYMS-inhibitor for the patient may be done if the patient harbors a variant listed in Table 1, at least one such additional marker, or both. In some of these embodiments at least one of these additional markers is chosen from Table 2.

In other embodiments the invention provides a computer-implemented method of determining whether a patient harbors a variant listed in Table 1 comprising: accessing the patient's genotype information stored in a computer-readable medium; querying this information to determine whether the patient has a variant listed in Table 1; and outputting [or displaying] said patient's genotype at the position corresponding to the variant. The method may optionally further output [or display] an indication that the patient's genotype is or is not associated with TYMS-inhibitor toxicity/sensitivity. Alternatively, the method may output [or display] an indication the patient has (or does not have) an increased likelihood of TYMS-inhibitor toxicity without displaying the patient's genotype.

In still another aspect the invention provides computer-implemented systems and methods involving the novel variants of the invention. In some embodiments the invention provides a computer-implemented method comprising:
(1) determining a patient's genotype at a position corresponding to a variant listed in Table 1 (including determining the amino acid in the patient's DPD protein at a position corresponding to a variant listed in Table 1) and inputting such information into a computer; and
(2) outputting [or displaying] the patient's genotype at this position.

In some embodiments the invention provides a computer-implemented treatment system comprising:
(1) determining whether a DPYD gene, DPYD mRNA or cDNA, or DPD protein (or a portion thereof) in a sample obtained from a patient harbors a variant listed in Table 1 and inputting such information into a computer;
(2) optionally determining whether said patient harbors any additional marker predictive of response and/or toxicity in treatment comprising a TYMS-inhibitor and inputting such information into a computer; and
(3) outputting (e.g., from a visual display generated by the computer) the conclusion that the patient has an increased likelihood of sensitivity or toxicity to treatment comprising a TYMS-inhibitor if the DPYD gene, DPYD mRNA or cDNA, or DPD protein (or a portion thereof) harbors a variant listed in Table 1.
   The computer may further or alternatively communicate that the patient should be monitored for toxicity, prescribed a treatment that does not comprise said TYMS-inhibitor, and/or have an initial dose of said TYMS-inhibitor adjusted if the patient harbors a variant listed in Table 1 and, optionally, at least one said additional marker.

In yet another aspect the invention provides a microarray comprising one or more isolated nucleic acids, proteins, or antibodies of the invention. In some embodiments the microarray comprises an oligonucleotide probe comprising at least one nucleotide variant listed in Table 1. In some embodiments the microarray comprises a peptide probe comprising at least one amino acid variant listed in Table 1. In yet other embodiments the microarray comprises an antibody probe that bind immunologically to a peptide comprising at least one amino acid variant listed in Table 1.

In still another aspect the invention provides a diagnostic kit comprising one or more isolated nucleic acids, proteins, or antibodies of the invention. In some embodiments the kit may additionally comprise: instructions for use, including instructions on interpreting the significance of the presence or absence of a variant listed in Table 1 (e.g., adjusting initial dose if the patient harbors such a variant); reagents useful for isolation, detection, amplification, quantification, and/or analysis of nucleic acids comprising, encompassing and/or containing a variant listed in Table 1; reagents useful for isolation, detection, quantification and/or analysis of peptides comprising, encompassing and/or containing a variant listed in Table 1 (including, e.g., antibodies that bind immunologically to such peptides); a microarray of the invention; etc.

The foregoing and other advantages and features of the invention, and the manner in which the same are accomplished, will become more readily apparent upon consideration of the following detailed description of the invention taken in conjunction with the accompanying examples and drawings, which illustrate preferred and exemplary embodiments.

### DETAILED DESCRIPTION OF THE INVENTION

The inventors have discovered several novel variants in the DPYD gene (Entrez Gene Id no. 1806) and its encoded DPD protein (SEQ ID NO:27; RefSeq Accession no. NP_{—}000101) that are associated with particular responses to treatment with thymidylate synthase (TYMS) inhibitors. The variants are depicted in detail in Table 1 below.

TABLE 1

| Variant (gene & | | SEQ ID |
|---|---|---|
| protein) | Sequence Context (gene & protein) | NO: |
| c.272G > T | cagatgccccgtgtcagaagagctTtccaactaatcttgatattaaatc | 64 |
| C91F | lgergalreamrclkcadapcqksFptnldiksfitsianknyygaakm | 84 |
| c.484-4G > A | ttaaccatgacaattgatttccccAtagGTATTCAAAGCAATGAGTATC | 79 |
| (IVS5-4G > A) | | |
| c.763-2A > G | cttatgcaaaatattgtttcttatGgATAATTTGCGGTAAAAGCCTTTC | 81 |
| (IVS7-2A > G) | | |
| c.1303A > G | gtccatctgaaagccgatgtggtcGtcagtgcctttggttcagttctga | 65 |
| I435V | teqdetgkwnededqmvhlkadvvVsafgsvlsdpkvkealspikfnrw | 85 |
| c.1337A > C | ttggttcagttctgagtgatcctaCagtaaaagaagccttgagccctat | 66 |
| K446T | dedqmvhlkadvvisafgsvlsdpTvkealspikfnrwglpevdpetmq | 86 |
| c.1349C > T | tgagtgatcctaaagtaaaagaagTcttgagccctataaaatttaacag | 67 |
| A450V | mvhlkadvvisafgsvlsdpkvkeVlspikfnrwglpevdpetmqtsea | 87 |
| c.1358C > T | ctaaagtaaaagaagccttgagccTtataaaatttaacagatggggtct | 68 |
| P453L | lkadvvisafgsvlsdpkvkealsLikfnrwglpevdpetmqtseawvf | 88 |
| c.1447G > A | tgggtatttgcaggtggtgatgtcAttggtttggctaacactacagtgg | 69 |
| V483I | wglpevdpetmqtseawvfaggdvIglanttvesvndgkqaswyihkyv | 89 |
| c.1552A > G | caatatggagcttccgtttctgccGagcctgaactacccctcttttaca | 70 |
| K518E | ndgkqaswyihkyvqsqqgasysaEpelplfytpidlvdisvemaglkf | 90 |
| c.1748T > C | ctttctctcttgataaggacattgCgacaaatgtttcccccagaatcat | 71 |
| V583A | mirrafeagwgfaltktfsldkdiAtnvspriirgttsgpmygpgqssf | 91 |
| c.1865G > A | gtgagaaaacggctgcatattggtAtcaaagtgtcactgaactaaaggc | 72 |
| C622Y | pmygpgqssflnielisektaaywYqsvtelkadfpdniviasimcsyn | 92 |
| c.2071G > T | gcctgtgggcaggatccagagctgTtgcggaacatctgccgctgggtta | 73 |
| V691L | lnlscphgmgergmglacgqdpelLrnicrwvrqavqipffakltpnvt | 93 |
| c.2482G > A | cagaatcaggatttcactgtgatcAaagactactgcactggcctcaaag | 74 |
| E828K | qflhsgasylqvcsaiqnqdftviKdyctglkallylksieelqdwdgq | 94 |
| c.2579A > - | agagtccagctactgtgagtcacc-gaaagggaaaccagttccacgtat | 78 |
| | iqnqdftviedyctglkallylksieelqdwdgqspatvshRKGNQFHV | 63 |
| c.2762T > A | tccccaaaaggcctattcctaccaAcaaggatgtaataggaaaagcact | 75 |
| I921N | lkeqnvafsplkrncfipkrpiptNkdvigkalqylgtfgelsnveqvv | 95 |
| c.2875T > C | gaaatgtgtatcaactgtggtaaaCgctacatgacctgtaatgattctg | 76 |
| C959R | fgelsnveqyvamideemcincgkRymtcndsgyqaiqfdpethlptit | 96 |
| c.2908-3C > T | atcaataccctctatttctgtttgTagGCTATACAGTTTGATCCAGAAA | 83 |
| (IVS22-3C > T) | | |
| c.2948C > T | cagaaacccacctgcccaccataaTcgacacttgtacaggctgtactct | 77 |
| T983I | cymtcndsgyqaiqfdpethlptiIdtctgctlclsvcpivdcikmvsr | 97 |

In Table 1, the uppercase bold letter in each oligonucleotide-oligopeptide pair corresponds to a novel variant of the invention. Each amino acid variant corresponds to (or may be encoded by) the nucleotide variant immediately above it. Each variant will be referred to individually by the variant name given in Table 1. For the sake of brevity, the amino acid variant will generally be given but, depending on the context, any discussion of an amino acid variant should be understood to apply equally to its corresponding nucleotide variant. The variant notation for polypeptides gives the position of each variant with respect to the consensus DPD protein sequence (SEQ ID NO:27; RefSeq Accession no. NP_{—}000101). The variant notation for nucleic acids gives the position of each variant with respect to the coding sequence (“CDS”) of the consensus DPYD cDNA (SEQ ID NO:100; RefSeq. Accession no. NM_{—}000110). The DPYD CDS (SEQ ID NO:101) consists of positions 138-3215 of SEQ ID NO:100. In the case of variants found in intronic sequences, lowercase type indicates intronic sequence while uppercase (non-bold) type indicates exonic sequence. Those skilled in the art are familiar with the notation formats provided for each variant, including the two alternative notations for each intronic variant. For example, c.272G>T indicates that a guanine is found at cDNA position 272 in the major allele while thymine is a variant (e.g., minor) allele at the same position. In the case of variant c.2579A>−, the deletion of adenine corresponding to CDS position 2579 results in a handful of amino acid sequence changes (bold, uppercase) followed by truncation of the protein after the new terminal valine residue. Thus SEQ ID NO:63 depicts the C-terminal end of the resulting truncated DPD protein.

Though not wishing to be bound by theory, the following is a discussion of the expected significance of each variant to DPD protein function and, by extension, to TYMS-inhibitor toxicity:
A. C91F: completely conserved down to C. elegans. The cysteine amino acid residue at this position is responsible for iron-sulfur binding, which is crucial for the stability and activity of the DPD protein.
B. c.484-4G>A (IVS5-4G>A): this intron position is close to the splice junction and could affect splice efficiency.
C. c.763-2A>G (IVS7-2A>G): this intron change directly affects the 3′ splice recognition site and thus probably leads to exon skipping.
D. I435V: completely conserved down to C. elegans. Part of a hydrophobic pocket together with Y304 and F309 that stabilizes a connection between two beta sheets and an alpha helix.
E. K446T: conserved in mouse, rat, pig and cow. Extends from the surface of the protein. Due its charged nature this residue could serve for regulatory protein-protein interactions.
F. A450V: completely conserved down to C. elegans. Inner side of an alpha-helix forming the outer boundary of the substrate binding pocket.
G. P453L: completely conserved down to C. elegans. P453 creates a turn in the peptide chain, bending the chain back towards the protein center. A leucine substitution is expected to interfere with that structural requirement. The closest amino acid that has a clear function in terms of coordinating a cofactor is T489 whose amide group stabilizes the N3 atom of FAD. T489 sits at the top of a long alpha helix. This long helix appears to be held in place by a loop in the peptide chain that is an extension of the turn starting with P453. Without P453 providing the required turn in the peptide chain, the entire bracket is not positioned properly and the arrangement of the whole helix could be destabilized.
H. V483I: conserved in mouse, rat, pig and cow.
I. K518E: not conserved. This residue does not appear to contact any functional domain or the dimer interface, but it extends from the protein surface and thus is expected to be primed to interact with another protein and, thus, could be essential for protein-protein interactions with a regulatory accessory protein.
J. V583A: completely conserved to C. elegans. Outer loop around the NADPH binding domain.
K. C622Y: conserved in mammals. Inner side of an alpha-helix forming the NADPH binding domain.
L. V691L: completely conserved to C. elegans. This residue lies at the end of an alpha helix that continues into the “active loop.” That loop is supposed to be flexible and lock the substrate in place for contacting the active site C671.
M. E828K: conserved in mouse, rat, fish and drosophila; Q in pig and cow; D in C. elegans. Deep inside the center of the protein this position appears to favor negatively charged amino acids since the residue interacts with K81 and/or K98 on an adjacent alpha-helix. K81 is located next to C82 and C79, two of the coordinating cysteines in the first N-terminal FeS cluster. Substitution with a negatively charged amino acid would disrupt the ionic interaction, disturb secondary structure in this area, and possibly affect stability of the iron-sulfur redox site.
N. 2579delA: frameshift results in truncation of the DPD protein at amino acid position 868, giving a polypeptide whose amino acid sequence comprises the sequence of SEQ ID NO:62.
O. I921N: not conserved, but hydrophobic (either I or V). Forms a hydrophobic pocket with L834, L806 and V535 that pulls together alpha-helices IVa7 and IVa8 with the beta sheet IVba.
P. C959R: completely conserved to C. elegans. The cysteine residue at this position is responsible for iron-sulfur binding, which is crucial for the stability and activity of the DPD protein.
Q. c.2908-3C>T (IVS22-3C>T): this intron position is next to the splice recognition site and could affect splicing efficiency. The preferred nucleotides at this position are C or A.
R. T983I: conserved in fish, pig, cow and C. elegans; S in mouse and rat. Thus preferring small, hydrophilic rather than large and hydrophobic side chains, possibly due to situation in a tight turn at the protein surface.

Those skilled in the art will recognize that these variants are expected to affect DPD protein function and, in turn, to affect how a patient with one or more of these variants in a DPYD gene responds to TYMS-inhibitors metabolized by DPD. Thus, the variants in Table 1 are useful in determining whether a patient will respond to or suffer toxicity from TYMS-inhibitors. As used herein, “TYMS-inhibitor” means a composition that inhibits the activity of the TYMS protein. This inhibition may be direct, as by binding the TYMS protein to inactivate it, or indirect, as by acting on the TYMS gene or mRNA to decrease expression of TYMS protein. One class of direct TYMS-inhibitor is the nucleotide analogs, including but not limited to 5-fluorouracil (5-FU).

Accordingly, one aspect of the invention provides isolated nucleic acids comprising at least one variant listed Table 1. As used herein, a nucleic acid or polypeptide “comprises” a variant if the nucleic acid or polypeptide contains or encompasses a residue corresponding to such variant within its linear sequence. A nucleic acid or polypeptide comprises a variant if the variant is found in any part of the linear sequence, including either end (e.g., the extreme 5′ or 3′ end in nucleic acids or the extreme N-terminal or C-terminal end in polypeptides).

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Specifically, since a naturally existing chromosome (or a viral equivalent thereof) includes a long nucleic acid sequence, an “isolated nucleic acid” as used herein means a nucleic acid molecule having only a portion of the nucleic acid sequence in the chromosome but not one or more other portions present on the same chromosome. More specifically, an “isolated nucleic acid” typically includes no more than 25 kb naturally occurring nucleic acid sequences which immediately flank the nucleic acid in the naturally existing chromosome (or a viral equivalent thereof). However, it is noted that an “isolated nucleic acid” as used herein is distinct from a clone in a conventional library such as genomic DNA library and cDNA library in that the clone in a library is still in admixture with almost all the other nucleic acids of a chromosome or cell. Thus, an “isolated nucleic acid” as used herein also should be substantially separated from other naturally occurring nucleic acids that are on a different chromosome of the same organism. Specifically, an “isolated nucleic acid” means a composition in which the specified nucleic acid molecule is significantly enriched so as to constitute at least 10% of the total nucleic acids in the composition. Often an isolated nucleic acid is synthetic, meaning it was synthesized in vitro or in an organism in which it is not naturally synthesized (e.g., in a genetically modified bacterium or yeast).

Some embodiments provide an isolated human gene, or a portion thereof, comprising a variant listed in Table 1. As used herein, “gene” refers to the entire DNA sequence-including exons, introns, and non-coding transcription-control regions-necessary for production of a functional protein or RNA. A “portion” of a gene will generally be a nucleic acid whose nucleotide sequence comprises (1) a contiguous stretch of nucleotides that is unique within the human genome to that gene (e.g., at least 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3500, 4000, 4500, 5000, 6000, 7000, 8000, 9000, 10000, 15000, 20000, 25000, 30000, 40000, 50000, 60000, 70000, 80000, 90000, 100000 or more contiguous nucleotides); and/or (2) a stretch of nucleotides of sufficient length and percent identity such that one skilled in the art would recognize the nucleic acid as coming from the gene or a variant of the gene rather than from an unrelated region of the genome (e.g., at least 20, 25, 30, 35, 40, 45, 50 or more nucleotides in length and at least 50%, 60%, 70%, 80%, 85%, 90%, 95%, or 99% identity). A “portion” of any other nucleic acid (e.g., mRNA, cDNA, oligonucleotide probe or primer, etc. that can serve as a reference sequence) is defined similarly (i.e., a nucleic acid whose nucleotide sequence comprises (1) a contiguous stretch of nucleotides that is unique within the human genome or transcriptome to that nucleic acid; and/or (2) a stretch of nucleotides of sufficient length and percent identity such that one skilled in the art would recognize the nucleic acid as coming from a variant of the nucleic acid rather than from an unrelated region of the genome or transcriptome).

Some embodiments provide isolated nucleic acids of various lengths comprising at least one variant of the invention. Such nucleic acids may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3000, 3500, 4000, 4500, 5000, 6000, 7000, 8000, 9000, 10000, 15000, 20000, 25000, 30000, 40000, 50000, 60000, 70000, 80000, 90000, 100000, 200000, 300000, 400000, 500000, 600000, 700000, 800000, 900000, 1000000 or more nucleotides in length or any range therein. Oligonucleotides (also called “oligos”) are relatively short nucleic acids and may be of any length listed above equal to or less than about 500. In some embodiments of the invention, oligos are between 5 and 500, 10 and 250, 18 and 150, 18 and 65, 22 and 250, 22 and 150, 22 and 65, or 23 and 65 nucleotides in length.

Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, or 4000 or more contiguous nucleotides of the sequence of SEQ ID NO:1, wherein the contiguous span comprises at least one variant listed in Table 1. Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350, 400, 450, 500, 600, 700, 800, 900, 1000, 1200, 1400, 1600, 1800, 2000, 2500, or 3000 or more contiguous nucleotides of the sequence of SEQ ID NO:26, wherein the contiguous span comprises at least one variant listed in Table 1.

Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 98, or 99 contiguous nucleotides of a sequence chosen from the group consisting of SEQ ID NOs 28-42, wherein the contiguous span comprises at least one variant listed in Table 1. Still other embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, or 79 contiguous nucleotides of a sequence chosen from the group consisting of SEQ ID NOs 43-47, wherein the contiguous span comprises at least one variant listed in Table 1. Still other embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, or 49 contiguous nucleotides of a sequence chosen from the group consisting of SEQ ID NOs 64-83, wherein the contiguous span comprises at least one variant listed in Table 1.

Those skilled in the art, apprised of the present disclosure, will be familiar with sequence analysis techniques for determining whether a variant listed in Table 1 is present in a particular nucleic acid or polypeptide—e.g., whether a thymine nucleotide in a test nucleic acid “corresponds” to the polymorphic thymine at position 272 of SEQ ID NO:26 or whether a phenylalanine residue in a test polypeptide “corresponds” to the polymorphic phenylalanine at position 91 of SEQ ID NO:2. Briefly, such techniques may include, but are not limited to: aligning the test sequence against one or more known DPYD or DPD sequences (e.g., SEQ ID NO:100 or SEQ ID NO:27); determining whether the test sequence has enough identity to one of these sequences to be a DPYD or DPD sequence (e.g., perfect alignment along a significant stretch or high enough percent identity to be recognized by those skilled in the art as DPYD or DPD or a portion or variant thereof); finding a nucleotide position in the test sequence that corresponds to one of the positions listed in Table 1; determining whether the test sequence has the variant residue listed in Table 1 for that position; contacting a sample with an antibody that selectively binds a DPD protein comprising at least one of the amino acid variants listed in Table 1; etc.

For the purpose of comparing two different nucleic acid or polypeptide sequences, one sequence (test sequence) may be described to be a specific “percentage identical to” another sequence (reference sequence) in the present disclosure. In this respect, the percentage identity may be determined by any algorithm known in the art, including but not limited to that of Karlin and Altschul, PROC. NATL. ACAD. Sci. USA, 90:5873-5877 (1993), which is incorporated into various BLAST programs. Specifically, the percentage identity may be determined by the “BLAST 2 Sequences” tool, which is available at NCBI's website. See Tatusova and Madden, FEMS MICROBIOL. LETT., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST 2.1.2., determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence.

In some embodiments the isolated nucleic acid of the invention comprises a variant listed in Table 1 at a particular position along its length. In some of these embodiments the variant residue is at the center of said isolated nucleic acid, In other embodiments the variant residue is within 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more nucleotide positions of the center of said isolated nucleic acid. In some embodiments the variant is no more than 5, no more than 4, no more than 3, no more than 2, or no more than 1 position from the center of the nucleic acid. As used herein, the “center” of a polynucleotide has the plain meaning given by those skilled in the art. The nucleotide (or pair of nucleotides) that, with respect to the linear sequence of nucleotides, has an equal number of residues on either side is the center of a polynucleotide. For instance, in the following oligonucleotide—5′-cagatgccccgtgtcagaagagctTtccaactaatcttgatattaaatc-3′ (SEQ ID NO:64)—the center of the oligo is the uppercase “T” residue because there are twenty-five residues on each side. Sometimes a polynucleotide has an even number of residues and thus the “center” is the pair of nucleotides that has an equal number of residues on either side of the pair. Sometimes those skilled in the art will be interested in the center of a relevant region of a nucleic acid rather than the center of the entire nucleic acid. For instance, an oligonucleotide probe or primer might comprise only a portion that hybridizes to a target nucleic acid (with the rest of the probe or primer free, in a hairpin loop, etc.). In such a case, one may refer to the “center” of the hybridizing portion of the oligonucleotide as the residue (or pair of residues) that has an equal number of hybridizing nucleotides on each side. Conversely, one may refer to the center of, e.g., the hairpin as the residue (or pair of residues) that has an equal number of hairpin nucleotides on each side.

In some embodiments the variant listed in Table 1 is within 0, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more nucleotide positions of the 5′ or 3′ end of a isolated nucleic acid of the invention. For example, the c.272G>T variant listed in Table 1 may appear at the extreme 5′ end of a nucleic acid of the invention, as in SEQ ID NO:98 (5′-Ttccaactaa tcttgatattaaatc-3′). As another example, the c.1303A>G variant listed in Table 1 may appear at the extreme 5′ end of a nucleic acid of the invention, as in SEQ ID NO:99 (5′-gtccatctgaaagccgatgtggtcG-3′).

In some embodiments the invention provides an isolated nucleic acid (e.g., an oligonucleotide) of the invention that selectively hybridizes to or amplifies a nucleic acid comprising a variant listed in Table 1. In some of these embodiments the isolated oligonucleotide hybridizes under stringent conditions to a nucleic acid whose nucleotide sequence consists of the sequence of SEQ ID NO:1 but not to a nucleic acid whose nucleotide sequence consists of the sequence of SEQ ID NO:100. In some embodiments this is accomplished by the oligo of the invention (1) encompassing a variant listed in Table 1 and (2) being of a such length and having the variant residue in such a position that the oligo will only hybridize under stringent (e.g., high stringency) conditions to nucleic acids that are highly homologous (sequence differences of 10%, 5%, 1% or less, including 0%).

The term “stringent conditions” is well-known in the art of nucleic acid hybridization and, as used herein, has its conventional meaning. The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringency hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

In some embodiments the isolated nucleic acid (e.g., an oligonucleotide) selectively amplifies (together with another primer, under standard conditions and with standard reagents) a nucleic acid whose nucleotide sequence comprises the sequence of SEQ ID NO:1, or a portion thereof, but not a nucleic acid whose nucleotide sequence comprises the sequence of SEQ ID NO:100, or a portion thereof. Often such a primer will, as above, only hybridize to nucleic acids with at least some minimum level of sequence identity (e.g., 90%, 95%, 96%, 97%, 98%, 99%, or 100%). Those skilled in the art are familiar with other ways of designing primers to only amplify certain sequences, often with single nucleotide specificity. As a non-limiting example, one may design a primer such that a variant listed in Table 1 is at or near the 3′ end of the primer (e.g., a primer comprising the sequence of SEQ ID NO:99). Thus, under stringent conditions the primer might hybridize to both wild-type and variant DPYD nucleic acids to some degree, while it's 3′ end will not hybridize unless the target nucleic acid is an exact match (e.g., the specific c.1303A>G DPYD variant).

In other embodiments of the present invention, isolated nucleic acids are provided which encode a contiguous span of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1025 or more amino acids of a DPD protein wherein said contiguous span contains at least one amino acid variant in Table 1 according to the present invention.

Some embodiments provide an isolated human protein or peptide, or a portion thereof, comprising a variant listed in Table 1. The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

A “portion” of a protein will generally be a polypeptide whose amino acid sequence comprises (1) a contiguous stretch of amino acids that is unique to that protein within the human proteome (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3500, 4000, 4500, 5000, 6000, 7000, 8000, 9000, 10000, 15000, 20000, 25000, 30000, 40000, 50000, 60000, 70000, 80000, 90000, 100000 or more contiguous amino acids); and/or (2) a stretch of amino acids of sufficient length and percent identity such that one skilled in the art would recognize the polypeptide as coming from a variant of the protein rather than from an unrelated protein (e.g., at least 20, 25, 30, 35, 40, 45, 50 or more amino acids in length and at least 50%, 60%, 70%, 80%, 85%, 90%, 95%, or 99% identity).

Some embodiments provide isolated polypeptides of various lengths comprising at least one variant of the invention. Such polypeptides may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1025 or more amino acids in length or any range therein. In some embodiments the polypeptide is any length listed above equal to or less than about 500. In other embodiments polypeptides are between 5 and 500, 8 and 250, 18 and 150, 18 and 65, 22 and 250, 22 and 150, 22 and 65, or 23 and 65 amino acids in length.

Some embodiments provide isolated polypeptides whose amino acid sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, or 1025 contiguous amino acids of the sequence of SEQ ID NO:2, wherein the contiguous span comprises at least one variant listed in Table 1. Some embodiments provide isolated polypeptides whose amino acid sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or 51 contiguous amino acids of the sequence of SEQ ID NOs 48-61, wherein the contiguous span comprises at least one variant listed in Table 1. Still other embodiments provide isolated nucleic acids whose nucleotide sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 49 contiguous amino acids of the sequence of SEQ ID NOs 63 & 84-97, wherein the contiguous span comprises at least one variant listed in Table 1.

Another aspect of the invention provides antibodies that bind specifically to a polypeptide variant of the invention and do not bind specifically to the wild-type DPD protein. Such antibodies may be generated based on the present novel sequence disclosures in Table 1 and various routine techniques known to those skilled in the art, such as those described in U.S. Pat. Nos. 5,837,492; 5,800,998 and 5,891,628. Antibodies may be raised against the proteins themselves or against peptide portions of the proteins. Such antibodies include, but are not limited to, polyclonal, monoclonal, F_{ab} fragments, F_{(ab′)2} fragments, single chain antibodies, chimeric antibodies, humanized antibodies etc. For example, antibodies that specifically bind variant DPD proteins can be raised by inoculating an animal with a peptide comprising one of the variants listed in Table 1 (optionally attached to some carrier molecule to increase immunogenicity if the peptide is small) and isolating the antibodies or the cells producing the antibodies from the animal. Example peptides include those depicted in SEQ ID NOs 84-97. The invention also provides hybridoma cell lines secreting antibodies of the invention.

Another aspect of the invention provides methods based on the variants listed in Table 1. These methods generally comprise determining whether a DPYD gene in a patient harbors a variant listed in Table 1. In some embodiments the method comprises determining whether a DPYD gene in a sample obtained from a patient harbors a variant chosen from the group consisting of: c.272G>T, C91F, c.484-4G>A (IVS5-4G>A), c.763-2A>G (IVS7-2A>G), c.1303A>G, I435V, c.1337A>C, K446T, c.1349C>T, A450V, c.1358C>T, P453L, c.1447G>A, V483I, c.1552A>G, K518E, c.1748T>C, V583A, c.1865G>A, C622Y, c.2071G>T, V691L, c.2482G>A, E828K, c.2579A>−, c.2762T>A, 1921N, c.2875T>C, C959R, c.2908-3C>T (IVS22-3C>T), c.2948C>T, and T983I. A “sample” is any biological specimen obtained from a patient or any substance derived therefrom. This may include tissue, solid tissue, bodily fluids (e.g., blood, serum, plasma, semen, saliva), waste products (e.g., urine, feces), etc. Substances of interest derived therefrom include, but are not limited to, nucleic acids or proteins (isolated and/or purified to any desired extent), small organic molecules (e.g., 5-FU or any metabolite thereof), cells or cell derivatives (e.g., exosomes, platelets), etc.

Determining whether a DPYD gene in a patient harbors the variants listed in Table 1 can be achieved by any technique known to those skilled in the art. Examples include, but are not limited to: (a) sequencing the DPYD gene (or a portion thereof) in a sample obtained from a patient; (b) sequencing the DPYD transcript (or a portion thereof) in a sample obtained from a patient; (c) determining the level of (including the presence or absence of) any nucleic acid or protein harboring a variant listed in Table 1.

In some embodiments germline DNA is analyzed (e.g., sequenced) to determine whether the gene harbors a variant. Detecting the level of any nucleic acid harboring a variant listed in Table 1 may be done by contacting a sample with oligonucleotides that selectively hybridize with nucleic acids harboring a variant listed in Table 1 (either free in solution or fixed to a substrate such as a microarray) or by subjecting a nucleic acid sample to conditions (e.g., reagents such as variant-specific primers) suitable for selective amplification of nucleic acids harboring a variant listed in Table 1.

Protein-based detection techniques may also prove to be useful, especially when the nucleotide variant causes amino acid substitutions or deletions or insertions or frameshift that affect the protein primary, secondary or tertiary structure. To detect the amino acid variations, protein sequencing techniques may be used. For example, HPLC-microscopy tandem mass spectrometry technique can be used for determining amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., ANAL. CHEM., 72:757-763 (2000). Other useful protein-based detection techniques include immunoaffinity assays based on antibodies selectively immunoreactive with mutant proteins according to the present invention. The method for producing such antibodies is described above in detail. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See, e.g., U.S. Pat. Nos. 4,376,110 and 4,486,530, both of which are incorporated herein by reference.

Additional variants that are in linkage disequilibrium (LD variants) with the nucleotide variants and/or haplotypes of the present invention can be identified by a haplotyping method known in the art, as will be apparent to a skilled artisan in the field of genetics and haplotying. The additional variants that are in linkage disequilibrium with a nucleotide variant in Table 1 can also be useful in the various applications as described below.

In some embodiments the invention provides a method of determining whether a patient has an increased likelihood of sensitivity and/or toxicity to treatment comprising a TYMS-inhibitor, the method comprising determining whether a DPYD gene in a sample obtained from the patient harbors a variant listed in Table 1, wherein the patient has an increased likelihood of sensitivity and/or toxicity to treatment comprising a TYMS-inhibitor if the DPYD gene harbors the variant.

As used herein, “increased likelihood of sensitivity” encompasses decreased likelihood of low sensitivity. Likewise, “increased likelihood of toxicity” encompasses decreased likelihood of low toxicity. Relative terms such as “increased, “decreased,” “high,” and “low” generally imply some reference or index level or value. Those skilled in the art are familiar with various techniques for determining such index values. The index value may represent the average (e.g., average sensitivity) in a plurality of training patients (e.g., both patients carrying a particular variant and patients not carrying such variant). For example, a “toxicity index value” can be generated from a plurality of training patients characterized as suffering toxicity from TYMS-inhibitor therapy. A “high sensitivity index value” can be generated from a plurality of training patients determined clinically to have high sensitivity. Thus, determining that a patient has a high or increased likelihood of toxicity to TYMS-inhibitor therapy, based at least in part on the patient's status for a variant listed in Table 1, can mean the patient's likelihood of toxicity is closer to the average patient determined clinically to have toxicity (or high toxicity) than to the average patient determined clinically to not have toxicity (or low toxicity). The same is true of high or increased likelihood of sensitivity to TYMS-inhibitor treatment.

Because information regarding a patient's likelihood of sensitivity/toxicity to treatment is important in choosing an appropriate course of treatment, the conclusion reached in practicing these methods of the invention (e.g., that the patient has an increased likelihood of sensitivity or toxicity to treatment comprising a TYMS-inhibitor) will often be recorded (such as in the patients health history, including electronic health records) and/or communicated to, e.g., a physician or other health care provider, the patient, etc. Thus the methods of the invention may further or optionally comprise recording that a patient has an increased likelihood of sensitivity or toxicity to treatment comprising a TYMS-inhibitor if the DPYD gene harbors a variant listed in Table 1.

In some embodiments the method further comprises determining whether the patient has any additional markers relevant to response or toxicity in TYMS-inhibitors. In some of these embodiments at least one of these additional markers is chosen from the group consisting of:

TABLE 2

| | Entrez |
|---|---|
| Gene | GeneId |
| Other | 1806 |
| DPYD | |
| variants | |
| TYMS | 7298 |
| UMPS | 7372 |
| TP53 | 7157 |
| DHFR | 1719 |
| MTHFR | 4524 |
| MTRR | 4552 |
| SLC19A1 | 6573 |
| SLC19A2 | 10560 |
| RRM1 | 6240 |
| RRM2 | 6241 |
| RRM2B | 50484 |
| ABCC11 | 85320 |
| ABCC5 | 10057 |
| DPYS | 1807 |
| SLC28A1 | 9154 |
| SLC28A2 | 9153 |
| SLC28A3 | 64078 |
| SLC29A1 | 2030 |
| SLC29A2 | 3177 |
| SLC29A3 | 55315 |
| SLC29A4 | 222962 |
| DKC1 | 1736 |

Yet another aspect of the invention provides treatment optimization methods based at least in part on whether a patient harbors a variant listed in Table 1. This aspect generally provides a method of optimizing TYMS-inhibitor treatment comprising:
(1) determining whether a DPYD gene, DPYD mRNA or cDNA, or DPD protein (or a portion thereof) in a sample obtained from a patient harbors a variant listed in Table 1; and
(2) administering, prescribing or recommending a specific treatment regimen based at least in part on whether the variant listed in Table 1.

In some embodiments the specific treatment regimen comprises: administering, prescribing or recommending a treatment that comprises a TYMS-inhibitor; adjusting the initial dose of a TYMS-inhibitor (e.g., adjusting the initial dose upward); and/or monitoring said patient for toxicity to treatment comprising a TYMS-inhibitor. Those skilled in the art are, based on the present disclosure, capable of administering, prescribing or recommending a treatment regimen comprising any combination of these.

In some embodiments the invention provides a method of optimizing TYMS-inhibitor treatment comprising:
(1) determining whether a DPYD gene, DPYD mRNA or cDNA, or DPD protein (or a portion thereof) in a sample obtained from a patient harbors a variant listed in Table 1;
(2) prescribing a low dose of a treatment that comprises a TYMS-inhibitor if the patient harbors the variant.
   As used herein, a “low dose” means some dose amount lower than the standard patient dose (including a weight-adjusted dose) for a treatment absent any indication the patient should have an altered dose. A standard dose may be the dose given to the average patient determined clinically to have optimal sensitivity (i.e., neither too high nor too low sensitivity) to a drug (e.g., 5-FU). A standard dose may include adjustments for the usual dose-adjustment criteria such as, e.g., a patient's age, weight, or general health, etc.

Thus, the invention provides treatment methods comprising administering, prescribing or recommending one or more of the above treatment regimens based at least in part on whether the patient harbors a variant listed in Table 1. Though the presence of a variant listed in Table 1 may be sufficient alone to justify any of the above treatment courses, physicians will often look to additional markers and/or clinical parameters in administering, prescribing or recommending a particular course of treatment for a particular patient. Thus the invention provides treatment methods comprising administering, prescribing or recommending one or more of the above treatment regimens based on (a) whether the patient harbors a variant listed in Table 1 and (b) the patient's status for at least one other marker (e.g., one or more of the additional markers listed above) or clinical parameter.

In some embodiments the invention provides a method of optimizing TYMS-inhibitor treatment comprising:
(1) determining whether a DPYD gene, DPYD mRNA or cDNA, or DPD protein (or a portion thereof) in a sample obtained from a patient harbors a variant listed in Table 1;
(2) determining whether the sample harbors any additional marker predictive of response and/or toxicity in treatment comprising a TYMS-inhibitor; and
(3) monitoring the patient for toxicity, prescribing a treatment that does not comprise the TYMS-inhibitor, and/or adjusting the initial dose of the TYMS-inhibitor for the patient if the patient harbors the variant and the additional marker.

In still another aspect the invention provides computer-implemented systems and methods involving the novel variants of the invention. In some embodiments the invention provides a computer-implemented method comprising:
(1) determining a patient's genotype at a position corresponding to a variant listed in Table 1 (including determining the amino acid in the patient's DPD protein at a position corresponding to a variant listed in Table 1) and inputting such information into a computer; and
(2) outputting [or displaying] the patient's genotype at this position.
   In some embodiments the patient's genotype at a position corresponding to a variant listed in Table 1 is displayed together with the canonical residue (e.g., the major allele) at this position, thereby allowing comparison to determine if the patient has a variant. In some embodiments the computer displays an indication that the patient harbors or does not harbor a variant at the position. In some embodiments the method further comprises displaying an indication that said patient has an increased likelihood of toxicity/sensitivity to TYMS-inhibitor treatment if the patient has a variant listed in Table 1.

In other embodiments the invention provides a computer-implemented method of determining whether a patient harbors a variant listed in Table 1 comprising: accessing the patient's genotype information stored in a computer-readable medium; querying this information to determine whether the patient has a variant listed in Table 1; and outputting [or displaying] said patient's genotype at the position corresponding to the variant. The method may optionally further output [or display] an indication that the patient's genotype is or is not associated with TYMS-inhibitor toxicity/sensitivity. Alternatively, the method may output [or display] an indication the patient has (or does not have) an increased likelihood of TYMS-inhibitor toxicity without displaying the patient's genotype.

As used herein, “genotype” has its conventional meaning in the art. Specifically in this context, a “patient's genotype information” means any information indicating the patient's genomic or mRNA (also cDNA) sequence (either germ-line or somatic) at any locus. “Locus” means any specific region of the patient's genome or transcriptome, including but not limited to, single nucleotide positions. As a non-limiting example, a patient's genotype at a position corresponding to position 25 in SEQ ID NO:64 is the nucleic acid residue (A, T, C, or G) at that position. As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

In some embodiments the invention provides a computer-implemented treatment system comprising:
(1) determining whether a DPYD gene, DPYD mRNA or cDNA, or DPD protein (or a portion thereof) in a sample obtained from a patient harbors a variant listed in Table 1 and inputting such information into a computer;
(2) optionally determining whether said patient harbors any additional marker predictive of response and/or toxicity in treatment comprising a TYMS-inhibitor and inputting such information into a computer; and
(3) outputting (e.g., from a visual display generated by the computer) the conclusion that the patient has an increased likelihood of sensitivity or toxicity to treatment comprising a TYMS-inhibitor if the DPYD gene, DPYD mRNA or cDNA, or DPD protein (or a portion thereof) harbors a variant listed in Table 1.
   The computer may further or alternatively communicate that the patient should be monitored for toxicity, prescribed a treatment that does not comprise said TYMS-inhibitor, and/or have an initial dose of said TYMS-inhibitor adjusted if the patient harbors a variant listed in Table 1 and, optionally, at least one said additional marker.

Another embodiment provides a method of determining whether a patient has a decreased likelihood of sensitivity to TYMS-inhibitor treatment comprising: accessing information on the patient's genotype stored in a computer-readable medium; querying this information to determine whether the patient harbors a variant listed in Table 1; outputting (or displaying) an indication that the patient has a decreased likelihood of sensitivity to TYMS-inhibitor treatment if the patient harbors the variant.

Determining whether a patient's DPYD gene, DPYD mRNA or cDNA, or DPD protein (or a portion thereof) harbors a variant listed in Table 1 can also be accomplished in silico (i.e., using a computer). In other words, an individual's genome sequence or sequences of a specific gene(s) (or genotype) or protein(s) may be already known, e.g., stored electronically in a computer-usable or computer-readable storage medium in a computer system or in a removable storage unit (e.g., floppy disks, magnetic tapes, optical disks, USB drives, and the like). Thus, by analyzing the sequence(s) in silico by computer (e.g., using an alignment program such as BLAST), one can determine the genotype at a particular locus, and determine if the individual has a variant listed in Table 1.

Typically, once a genotype at a locus or the presence or absence of a variant listed in Table 1 is determined or the disease diagnosis or prognosis correlating to the genotype is made, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a variant listed in Table 1 in the individual tested can be embodied in descriptive statements, diagrams, photographs, charts, images or any other visual forms. For example, images of gel electrophoresis of PCR products can be used in explaining the results. Diagrams showing where the variant listed in Table 1 occurs in an individual genome are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a variant listed in Table 1 in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on a genotype of an individual. The method comprises the steps of (1) determining the presence or absence of a nucleotide variant according to the present invention in the genome of the individual; and (2) embodying the result of the determining step in a transmittable form. The transmittable form is a product of the production method.

In yet another aspect the invention provides a microarray comprising one or more isolated nucleic acids of the invention. As is known in the art, in microchips, a large number of different nucleic acid probes are attached or immobilized in an array on a solid support, e.g., a silicon chip or glass slide. Target nucleic acid sequences to be analyzed can be contacted with the immobilized oligonucleotide probes on the microchip. See Lipshutz et al., BIOTECHNIQUES, 19:442-447 (1995); Chee et al., SCIENCE, 274:610-614 (1996); Kozal et al., NAT. MED., 2:753-759 (1996); Hacia et al., NAT. GENET., 14:441-447 (1996); Saiki et al., PROC. NATL. ACAD. SCI. USA, 86:6230-6234 (1989); Gingeras et al., GENOME RES., 8:435-448 (1998). The microchip technologies combined with computerized analysis tools allow large-scale high throughput screening. See, e.g., U.S. Pat. No. 5,925,525 to Fodor et al; Wilgenbus et al., J. MOL. MED., 77:761-786 (1999); Graber et al., CURR. OPIN. BIOTECHNOL., 9:14-18 (1998); Hacia et al., NAT. GENET., 14:441-447 (1996); Shoemaker et al., NAT. GENET., 14:450-456 (1996); DeRisi et al., NAT. GENET., 14:457-460 (1996); Chee et al., NAT. GENET., 14:610-614 (1996); Lockhart et al., NAT. GENET., 14:675-680 (1996); Drobyshev et al., GENE, 188:45-52 (1997).

In some embodiments the microarray comprises oligonucleotide probes comprising a variant listed in Table 1. In one embodiment, a DNA microchip is provided having a plurality of from 2 to 2,000,000 or more oligonucleotides, or from 10 to 600,000, or from 500 to 500,000, or from 1,000 to 50,000 oligonucleotides. In some embodiments, each microchip includes at least 2, 3, 4, 5, 6, 7, 8, 9, 10, 12, 15, 20, 25, 30, 40 or 50, or at least 70, 80, 90 or 100 or more variant-containing oligonucleotides of the present invention, each comprising a variant listed in Table 1. In specific embodiments, the nucleotide sequence of each of the variant-containing oligonucleotides comprises at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 98, or 99 contiguous nucleotides of a sequence chosen from the group consisting of SEQ ID NOs 28-42, wherein the contiguous span comprises at least one variant listed in Table 1. In other embodiments, the nucleotide sequence of each of the variant-containing oligonucleotides comprises at least 18, 19, 20, 21, 22, 23, 24, 25, 26, 27, 28, 29, 30, 31, 32, 33, 34, 35, 36, 37, 38, 39, 40, 45, 50, 60, 70, or 79 contiguous nucleotides of a sequence chosen from the group consisting of SEQ ID NOs 43-47, wherein the contiguous span comprises at least one variant listed in Table 1. In still other embodiments, the nucleotide sequence of the variant-containing oligonucleotides comprises at least 18, 19, 20, 21, 22, 23, 24, 25, 26, 27, 28, 29, 30, 31, 32, 33, 34, 35, 36, 37, 38, 39, 40, 45, or 49 contiguous nucleotides of a sequence chosen from the group consisting of SEQ ID NOs 64-83, wherein the contiguous span comprises at least one variant listed in Table 1

In still another aspect the invention provides a kit for genotyping, i.e., determining the presence or absence of one or more of the nucleotide or amino acid variants of present invention in the genomic DNA, or cDNA or mRNA in a sample obtained from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques. The kit may comprise one or more isolated nucleic acids of the invention. The kit may comprise a protein, peptide and/or antibody of the invention. In some embodiments the kit may additionally comprise: instructions for use, including instructions on interpreting the significance of the presence or absence of a variant listed in Table 1 (e.g., adjusting initial or subsequent doses if the patient harbors a variant); reagents needed for isolation, detection, and/or amplification of nucleic acids comprising a variant of the invention; a microarray of the invention; etc.

In one embodiment, the detection kit includes one or more oligonucleotides useful in detecting one or more of the nucleotide variants in Table 1, or an LD variant thereof. The oligonucleotides can be in one or more compartments or containers in the kit. In one embodiment, the kit has a plurality of from 2 to 2000 oligonucleotides, or from 5 to 2000, or from 10 to 2000, or from 25 or 50 to 500, 1000, 1500 or 2000 oligonucleotides. In one embodiment, each kit includes at least 2, 3, 4, 5, 6, 7, 8, 9, 10, 12, 15, 20, 25, 30, 40 or 50, or at least 70, 80, 90 or 100 variant-containing oligonucleotides of the present invention, each comprising a variant selected from those in Table 1, or the complement thereof. In some embodiments, each of the variant-containing oligonucleotides comprises a contiguous span of at least 12, 15, 17, 18, 19, 20, 21, 22, 23, 24, 25, 26, 27, 28, 29, 30, 40, 50, 60, 70, 80, 90, or 99 nucleotide residues of any one of SEQ ID NOs:28-42, and each contains at least one nucleotide variant of those in Table 1, or the complement thereof. In some embodiments, each variant-containing oligonucleotide has a contiguous span of from about 17, 18, 19, 20, 21, 22, 23 or 25 to about 30, 40 or 50, preferably from about 21 to about 30, 40, 50 or 60 nucleotide residues, of any one of SEQ ID NOs:28-42, containing one nucleotide variant selected from those in Table 1, or the complement thereof.

In the kit of the present invention having oligonucleotides, the oligonucleotides can be affixed to a solid support, e.g., incorporated in a microchip or microarray included in the kit. In other words, microchips and microarrays according to the present invention described above in Section 3 can be included in the kit.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Alternatively, the oligonucleotides included in the kit are not labeled, and instead, one or more markers are provided in the kit so that users may label the oligonucleotides at the time of use.

In another embodiment of the invention, the detection kit contains one or more antibodies that bind selectively to certain protein variants containing specific amino acid variants of the invention.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, mutS protein, and the like. In addition, the detection kit preferably includes instructions on using the kit for detecting nucleotide variants in human samples.

### Example

Novel variants in DPD were identified in cancer patients. 676 cancer patient samples were sequenced. All exons and the proximal promoter of the DPYD gene were PCR™ amplified using exon-specific primers and PCR™ products were sequenced by dye-primer chemistry. The following variants were identified:

TABLE 3

| Nucleotide | Amino Acid Variant |
|---|---|
| Variant | (if applicable) |
| c.272G > T | C91F |
| c.484-4G > A | |
| (IVS5-4G > A) | |
| c.763-2A > G | |
| (IVS7-2A > G) | |
| c.1303A > G | I435V |
| c.1337A > C | K446T |
| c.1349C > T | A450V |
| c.1358C > T | P453L |
| c.1447G > A | V483I |
| c.1552A > G | K518E |
| c.1748T > C | V583A |
| c.1865G > A | C622Y |
| c.2071G > T | V691L |
| c.2482G > A | E828K |
| c.2579A>− | |
| c.2762T > A | I921N |
| c.2875T > C | C959R |
| c.2908-3C > T | |
| (IVS22-3C > T) | |
| c.2948C > T | T983I |

Conservation was derived from an amino acid alignment of DPD proteins from mouse, rat, cow, pig, fish, drosophila and C. elegans. Functionality of variants was inferred above from the crystal structure of DPD published in Dobritzsch et al., EMBO J. (2001) 20:650-660.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid at least 18 nucleotides in length encoding at least one amino acid variant listed in Table 1, or the complement thereof.

2. The isolated nucleic acid of claim 1 comprising at least 18 consecutive nucleotides of any one of SEQ ID NOs 1, 26, & 102-104 wherein said at least 18 consecutive nucleotides comprise at least one of the nucleotide variants listed in Table 1, or the complement thereof.

3. An isolated polypeptide comprising at least 8 consecutive amino acids of SEQ ID NO:2, wherein said at least 8 consecutive amino acids comprise at least one of the amino acid variants listed in Table 1.

4. An isolated antibody the binds specifically to the isolated polypeptide of claim 3.

5-11. (canceled)

12. A kit comprising reagents suitable for detecting at least one of the variants listed in Table 1.

13. The kit of claim 12, wherein said reagents comprise oligonucleotide primers suitable for selectively amplifying a DPYD nucleic acid having a variant listed in Table 1.

14. The kit of claim 12, wherein said reagents comprise at least one oligonucleotide probe that specifically hybridizes under stringent conditions to said at least one variant.

15. The kit of claim 14 having a plurality of said probes fixed to at least one solid support.

16. The kit of claim 12, wherein said reagents comprise at least one antibody that specifically binds a DPD protein having said at least one variant.


SEQUENCE LISTING (104 sequences)

SEQ ID NO: 1 - DNA, 4451 bp, Homo sapiens
  misc_feature (138)..(3215): CDS from position 138 to position 3215
gctccgcccccgcgccgccggccctagtctgcctgttttcgactcgcgctccggctgctgtcacttggctctctggctggagcttgaggacgcaaggagggtttgtcactggcagactcgagactgtaggcactgccatggcccctgtgctcagtaaggactcggcggacatcgagagtatcctggctttaaatcctcgaacacaaactcatgcaactctgtgttccacttcggccaagaaattagacaagaaacattggaaaagaaatcctgataagaactgctttaattgtgagaagctggagaataattttgatgacatcaagcacacgactcttggtgagcgaggagctctccgagaagcaatgagatgcctgaaatgtgcagatgccccgtgtcagaagagctttccaactaatcttgatattaaatcattcatcacaagtattgcaaacaagaactattatggagctgctaagatgatattttctgacaacccacttggtctgacttgtggaatggtatgtccaacctctgatctttgtgtaggtggatgcaatttatatgccactgaagagggacccattaatattggtggattgcagcaatttgctactgaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaagtacttctgaaattcctcagttccggctgccgtatgatgtagtgaattttgagattgagctaatgaaggaccttggtgtaaagataatttgcggtaaaagcctttcagtgaatgaaatgactcttagcactttgaaagaaaaaggctacaaagctgctttcattggaataggtttgccagaacccaataaagatgccatcttccaaggcctgacgcaggaccaggggttttatacatccaaagactttttgccacttgtagccaaaggcagtaaagcaggaatgtgcgcctgtcactctccattgccatcgatacggggagtcgtgattgtacttggagctggagacactgcctttgactgtgcaacatctgctctacgttgtggagctcgccgtgtgttcatcgtcttcagaaaaggctttgttaatataagagctgtccctgaggagatggaacttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcgtcagtgcctttggttcagttctgagtgatcctacagtaaaagaagtcttgagccttataaaatttaacagatggggtctcccagaagtagatccagaaactatgcaaactagtgaagcatgggtatttgcaggtggtgatgtcattggtttggctaacactacagtggaatcggtgaatgatggaaagcaagcttcttggtacattcacaaatacgtacagtcacaatatggagcttccgtttctgccgagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggacattgcgacaaatgtttcccccagaatcatccggggaaccacctctggccccatgtatggccctggacaaagctcctttctgaatattgagctcatcagtgagaaaacggctgcatattggtatcaaagtgtcactgaactaaaggctgactttccagacaacattgtgattgctagcattatgtgcagttacaataaaaatgactggacggaacttgccaagaagtctgaggattctggagcagatgccctggagttaaatttatcatgtccacatggcatgggagaaagaggaatgggcctggcctgtgggcaggatccagagctgttgcggaacatctgccgctgggttaggcaagctgttcagattcctttttttgccaagctgaccccaaatgtcactgatattgtgagcatcgcaagagctgcaaaggaaggtggtgccaatggcgttacagccaccaacactgtctcaggtctgatgggattaaaatctgatggcacaccttggccagcagtggggattgcaaagcgaactacatatggaggagtgtctgggacagcaatcagacctattgctttgagagctgtgacctccattgctcgtgctctgcctggatttcccattttggctactggtggaattgactctgctgaaagtggtcttcagtttctccatagtggtgcttccgtcctccaggtatgcagtgccattcagaatcaggatttcactgtgatcaaagactactgcactggcctcaaagccctgctttatctgaaaagcattgaagaactacaagactgggatggacagagtccagctactgtgagtcaccagaaagggaaaccagttccacgtatagctgaactcatggacaagaaactgccaagttttggaccttatctggaacagcgcaagaaaatcatagcagaaaacaagattagactgaaagaacaaaatgtagctttttcaccacttaagagaaactgttttatccccaaaaggcctattcctaccaacaaggatgtaataggaaaagcactgcagtaccttggaacatttggtgaattgagcaacgtagagcaagttgtggctatgattgatgaagaaatgtgtatcaactgtggtaaacgctacatgacctgtaatgattctggctaccaggctatacagtttgatccagaaacccacctgcccaccataatcgacacttgtacaggctgtactctgtgtctcagtgtttgccctattgtcgactgcatcaaaatggtttccaggacaacaccttatgaaccaaagagaggcgtacccttatctgtgaatccggtgtgttaaggtgatttgtgaaacagttgctgtgaactttcatgtcacctacatatgctgatcttttaaaatcatgatccttgtgttcagctctttccaaattaaaacaaatatacattttctaaataaaaatatgtaatttcaaaatacatttgtaagtgtaaaaaatgtctcatgtcaatgaccattcaattagtggtcataaaatagaataattcttttctgaggatagtagttaaataactgtgtggcagttaattggatgttcactgccagttgtcttatgtgaaaaattaacttttttgtggcaattagtgtgacagtttccaaattgccctatgctgtgctccatatttgatttctaattgtaagtgaaattaagcattttgaaacaaagtactctttaacatacaagaaaatgtatccaaggaaacattttatcattaaaaattacctttaattttaatgctgtttctaagaaaatgtagttagctccataaagtacaaatgaagaaagtcaaaaaattatttgctatggcaggataagaaagcctaaaattgagtttgtagaactttattaagtaaaatccccttcgctgaaattgcttatttttggtgttggatagaggatagggagaatatttactaactaaataccattcactactcatgcgtgagatgggtgtacaaactcatcctcttttaatggcatttctctttaaactatgttcctaacaaaatgagatgataggatagatcctggttaccactcttttgctgtgcacatacgggctctgactggttttaatagtcaccttcatgattatagcaactaatgtttgaacaaagctcaaagtatgcaatgcttcattattcaagaatgaaaaatataatgttgataatatatattaagtgtgccaaatcagtttgactactctctgttttagtgtttatgtttaaaagaaatatattttttgttattattagataatatttttgtatttctctattttcataatcagtaaatagtgtcatataaactcatttatctcctcttcatggcatcttcaatatgaatctataagtagtaaatcagaaagtaacaatctatggcttatttctatgacaaattcaagagctagaaaaataaaatgtttcattatgcacttttagaaatgcatatttgccacaaaacctgtattactgaataatatcaaataaaatatcataaagcattttaaaaaa

SEQ ID NO: 2 - protein, 1025 aa, Homo sapiens
MAPVLSKDSADIESILALNPRTQTHATLCSTSAKKLDKKHWKRNPDKNCFNCEKLENNFDDIKHTTLGERGALREAMRCLKCADAPCQKSFPTNLDIKSFITSIANKNYYGAAKMIFSDNPLGLTCGMVCPTSDLCVGGCNLYATEEGPINIGGLQQFATEVFKAMSIPQIRNPSLPPPEKMSEAYSAKIALFGAGPASISCASFLARLGYSDITIFEKQEYVGGLSTSEIPQFRLPYDVVNFEIELMKDLGVKIICGKSLSVNEMTLSTLKEKGYKAAFIGIGLPEPNKDAIFQGLTQDQGFYTSKDFLPLVAKGSKAGMCACHSPLPSIRGVVIVLGAGDTAFDCATSALRCGARRVFIVFRKGFVNIRAVPEEMELAKEEKCEFLPFLSPRKVIVKGGRIVAMQFVRTEQDETGKWNEDEDQMVHLKADVVVSAFGSVLSDPTVKEVLSLIKFNRWGLPEVDPETMQTSEAWVFAGGDVIGLANTTVESVNDGKQASWYIHKYVQSQYGASVSAEPELPLFYTPIDLVDISVEMAGLKFINPFGLASATPATSTSMIRRAFEAGWGFALTKTFSLDKDIATNVSPRIIRGTTSGPMYGPGQSSFLNIELISEKTAAYWYQSVTELKADFPDNIVIASIMCSYNKNDWTELAKKSEDSGADALELNLSCPHGMGERGMGLACGQDPELLRNICRWVRQAVQIPFFAKLTPNVTDIVSIARAAKEGGANGVTATNTVSGLMGLKSDGTPWPAVGIAKRTTYGGVSGTAIRPIALRAVTSIARALPGFPILATGGIDSAESGLQFLHSGASVLQVCSAIQNQDFTVIKDYCTGLKALLYLKSIEELQDWDGQSPATVSHQKGKPVPRIAELMDKKLPSFGPYLEQRKKIIAENKIRLKEQNVAFSPLKRNCFIPKRPIPTNKDVIGKALQYLGTFGELSNVEQVVAMIDEEMCINCGKRYMTCNDSGYQAIQFDPETHLPTIIDTCTGCTLCLSVCPIVDCIKMVSRTTPYEPKRGVPLSVNPVC

SEQ ID NO: 3 - DNA, 276 bp, Homo sapiens
  misc_feature (51)..(226): Exon 1
gcaggaccgagagcgcagccccgccccgggggcgttgccgccccgcgcccgctccgcccccgcgccgccggccctagtctgcctgttttcgactcgcgctccggctgctgtcacttggctctctggctggagcttgaggacgcaaggagggtttgtcactggcagactcgagactgtaggcactgccatggcccctgtgctcagtaaggactcggcggacatcgaggtacggacttcgcgccggctcgtcggtggtggcggggtggtggggagtgc

SEQ ID NO: 4 - DNA, 211 bp, Homo sapiens
  misc_feature (51)..(161): Exon 2
aaatgccaacatatttccatatatttacacattgtttatgctgtctttagagtatcctggctttaaatcctcgaacacaaactcatgcaactctgcgttccacttcggccaagaaattagacaagaaacattggaaaagaaatcctgataagaactgctttgtaagtaccactgatacactatttcatgctgaaaattacctcactccaca

SEQ ID NO: 5 - DNA, 183 bp, Homo sapiens
  misc_feature (51)..(133): Exon 3
atttttctcaggatcttagagaattaatgcaatttttaaatatgttgcagaattgtgagaagctggagaataattttgatgacatcaagcacacgactcttggtgagcgaggagctctccgagaagcaatgaggtaagtctgggtcaccatagcaacagtcacagatgtggtaaagaaaaagt

SEQ ID NO: 6 - DNA, 188 bp, Homo sapiens
  misc_feature (51)..(138): Exon 4
attatctcacttatcttgtagtttcttaacatagttattttatttcctagatgcctgaaatgtgcagatgccccgtgtcagaagagctgtccaactaatcttgatattaaatcattcatcacaagtattgcaaacaaggtaaattcagatttaactctgcaaatgaaaataacagtatttgatcttgt

SEQ ID NO: 7 - DNA, 262 bp, Homo sapiens
  misc_feature (51)..(212): Exon 5
ttaaagtatatttttaagtagtaaaattttattttgtattaattttgcagaactattatggagctgctaagatgatattttctgacaacccacttggtctgacttgtggaatggtatgtccaacctctgatctttgtgtaggtggatgcaatttatatgccactgaagagggacccattaatattggtggattgcagcaatttgctactgaggtatgtatgatatacacgtgacatccccccactaccatcaccatgcacaa

SEQ ID NO: 8 - DNA, 297 bp, Homo sapiens
  misc_feature (51)..(247): Exon 6
gatgtaagctagtttcaaaattttaaccatgacaattgatttccccgtaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaaggtaatgcctacatttatttcatgtttatagtgtcagaaatgatggagcaa

SEQ ID NO: 9 - DNA, 182 bp, Homo sapiens
  misc_feature (51)..(132): Exon 7
attggtcatttttctactgatgcctgtttttctttctcctttcttttcagtacttctgaaattcctcagttccggctgccgtatgatgtagtgaattttgagattgagctaatgaaggaccttggtgtaaaggtaaatgaaaaaaacacctatctgtgtactgctcaaaaagaaaggagtaa

SEQ ID NO: 10 - DNA, 188 bp, Homo sapiens
  misc_feature (51)..(138): Exon 8
ttaattaatcatatgccaaattttcttatgcaaaatattgtttcttatagataatttgcggtaaaagcctttcagtgaatgaaatgactcttagcactttgaaagaaaaaggctacaaagctgctttcattggaataggtgagtagtttactcttaaatatatttatttataatgtatttttttattt

SEQ ID NO: 11 - DNA, 208 bp, Homo sapiens
  misc_feature (51)..(158): Exon 9
ggaaaagcccctcctcctgctaatgatattttataaatttgattacttaggtttgccagaacccaataaagatgccatcttccaaggcctgacgcaggaccaggggttttatacatccaaagactttttgccacttgtagccaaaggcagtaaagcaggtataacatattgtcttttatgcttaatagtgagtatgctattttattta

SEQ ID NO: 12 - DNA, 270 bp, Homo sapiens
  misc_feature (51)..(220): Exon 10
gcagaaatggtttctctacttgaaaatggggttttcctttcatcattcaggaatgtgcgcctgtcactctccattgccatcgatacggggagtcgtgattgtacttggagctggagacactgcctttgactgtgcaacatctgctctacgttgtggagctcgccgtgtgttcatcgtcttcagaaaaggctttgttaatataagagctgtccctgaggaggtaaaatggaaccatcagaaaatatggagttgtactccaacagattttag

SEQ ID NO: 13 - DNA, 311 bp, Homo sapiens
  misc_feature (51)..(261): Exon 11
gtggaataagaaacattttcaagtttcttctctgttctgttttgttttagatggaacttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcatcagtgcctttggttcagttctgagtgatcctaaaggtacagtgctgggagctgaaatgtgtgatgcaattgtctgttattttagt

SEQ ID NO: 14 - DNA, 285 bp, Homo sapiens
  misc_feature (51)..(235): Exon 12
aattgtctgttgactagtgattgtctattgtttttcacttgttttttcagtaaaagaagccttgagccctataaaatttaacagatggggtctcccagaagtagatccagaaactatgcaaactagtgaagcatgggtatttgcaggtggtgatgtcgttggtttggctaacactacagtggaatcggtgaatgatggaaagcaagcttcttggtacattcacaaatacgtacaggtaggcatttgccatcatttccacttaattcttttccaatggataagtgc

SEQ ID NO: 15 - DNA, 316 bp, Homo sapiens
  misc_feature (51)..(266): Exon 13
tggacaatttagatgtaatatgaaaccaagtattggtttgtattttgcagtcacaatatggagcttccgtttctgccaagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggtaagaaaatattattgaagtcatatagaaatgtctatcatatattttaa

SEQ ID NO: 16 - DNA, 265 bp, Homo sapiens
  misc_feature (51)..(215): Exon 14
atagcttttctttgtcaaaaggagactcaatatctttactctttcatcaggacattgtgacaaatgtttcccccagaatcatccggggaaccacctctggccccatgtatggccctggacaaagctcctttctgaatattgagctcatcagtgagaaaacggctgcatattggtgtcaaagtgtcactgaactaaaggctgactttccagacaacgtaagtgtgatttaacatctaaaacaagagaattggcataagttggtgaa

SEQ ID NO: 17 - DNA, 169 bp, Homo sapiens
  misc_feature (51)..(119): Exon 15
acaactggataactacttgaacttatattcttttgtttttctttttaaagattgtgattgctagcattatgtgcagttacaataaaaatgactggacggaacttgccaagaagtctgaggtaatggttactttagtatatatttattttatttcttctctcctattttt

SEQ ID NO: 18 - DNA, 184 bp, Homo sapiens
  misc_feature (51)..(134): Exon 16
tatataaataaagctttgccttttctttcattttcctttcttgtttaaaggattctggagcagatgccctggagttaaatttatcatgtccacatggcatgggagaaagaggaatgggcctggcctgtgggcaggtaaggaccttgacagctgccactaagtatgtcagtgtgacataacaggc

SEQ ID NO: 19 - DNA, 221 bp, Homo sapiens
  misc_feature (51)..(171): Exon 17
gatcacaattctttaattttgttctatatttaaccgacctatttgaacaggatccagagctggtgcggaacatctgccgctgggttaggcaagctgttcagattcctttttttgccaagctgaccccaaatgtcactgatattgtgagcatcgcaagagctgcaaaggaaggtaagaacttgacttgaatcagttgcccgctattgtaaatattggcccac

SEQ ID NO: 20 - DNA, 220 bp, Homo sapiens
  misc_feature (51)..(170): Exon 18
ctatcgtgtcttataagagctgcatgaaaatgttgatgtgtcttgcataggtggtgccaatggcgttacagccaccaacactgtctcaggtctgatgggattaaaatctgatggcacaccttggccagcagtggggattgcaaagcgaactacatatggaggagtgtctggtaggtgttgcccacttcttgcattgtgctttcttggaggttgctgaaaa

SEQ ID NO: 21 - DNA, 243 bp, Homo sapiens
  misc_feature (51)..(193): Exon 19
atactcaagtggtcagtgtgctaaccattgattattctgattttgtgtagggacagcaatcagacctattgctttgagagctgtgacctccattgctcgtgctctgcctggatttcccattttggctactggtggaattgactctgctgaaagtggtcttcagtttctccatagtggtgcttccgtcctccaggtagtcattgtgtttgtctgtcccttttaaaaatctccatctcacaaatg

SEQ ID NO: 22 - DNA, 280 bp, Homo sapiens
  misc_feature (51)..(230): Exon 20
gagaattatctcttggtttgacacttggttgactgctctgtcttttctaggtatgcagtgccattcagaatcaggatttcactgtgatcgaagactactgcactggcctcaaagccctgctttatctgaaaagcattgaagaactacaagactgggatggacagagtccagctactgtgagtcaccagaaagggaaaccagttccacgtatagctgaactcatggacaaggtatgtgcttaaatctcctgtgttattcttgagaactcagccttggtttc

SEQ ID NO: 23 - DNA, 244 bp, Homo sapiens
  misc_feature (51)..(194): Exon 21
ttctcttctctgagctaacatgcttccttatttgtgtgttttccttttagaaactgccaagttttggaccttatctggaacagcgcaagaaaatcatagcagaaaacaagattagactgaaagaacaaaatgtagctttttcaccacttaagagaaactgttttatccccaaaaggcctattcctaccatcaaggtaaaaattatgccaactatataagtatgcctactttactggttaaatat

SEQ ID NO: 24 - DNA, 241 bp, Homo sapiens
  misc_feature (51)..(191): Exon 22
tgaaaatgttttaaaatgagcttgctaagtaattcagtggctatttttaggatgtaataggaaaagcactgcagtaccttggaacatttggtgaattgagcaacgtagagcaagttgtggctatgattgatgaagaaatgtgtatcaactgtggtaaatgctacatgacctgtaatgattctggctaccaggtaagaatcctgctggaattagaatgctatgagacatgtttcttcttgtt

SEQ ID NO: 25 - DNA, 1503 bp, Homo sapiens
  misc_feature (51)..(1453): Exon 23
tgttgacctttgtggtcagtgacatcaataccctctatttctgtttgcaggctatacagtttgatccagaaacccacctgcccaccataaccgacacttgtacaggctgtactctgtgtctcagtgtttgccctattgtcgactgcatcaaaatggtttccaggacaacaccttatgaaccaaagagaggcgtacccttatctgtgaatccggtgtgttaaggtgatttgtgaaacagttgctgtgaactttcatgtcacctacatatgctgatcttttaaaatcatgatccttgtgttcagctctttccaaattaaaacaaatatacattttctaaataaaaatatgtaatttcaaaatacatttgtaagtgtaaaaaatgtctcatgtcaatgaccattcaattagtggtcataaaatagaataattcttttctgaggatagtagttaaataactgtgtggcagttaattggatgttcactgccagttgtcttatgtgaaaaattaacttttttgtggcaattagtgtgacagtttccaaattgccctatgctgtgctccatatttgatttctaattgtaagtgaaattaagcattttgaaacaaagtactctttaacatacaagaaaatgtatccaaggaaacattttatcattaaaaattacctttaattttaatgctgtttctaagaaaatgtagttagctccataaagtacaaatgaagaaagtcaaaaaattatttgctatggcaggataagaaagcctaaaattgagtttgtagaactttattaagtaaaatccccttcgctgaaattgcttatttttggtgttggatagaggatagggagaatatttactaactaaataccattcactactcatgcgtgagatgggtgtacaaactcatcctcttttaatggcatttctctttaaactatgttcctaacaaaatgagatgataggatagatcctggttaccactcttttgctgtgcacatacgggctctgactggttttaatagtcaccttcatgattatagcaactaatgtttgaacaaagctcaaagtatgcaatgcttcattattcaagaatgaaaaatataatgttgataatatatattaagtgtgccaaatcagtttgactactctctgttttagtgtttatgtttaaaagaaatatattttttgttattattagataatatttttgtatttctctattttcataatcagtaaatagtgtcatataaactcatttatctcctcttcatggcatcttcaatatgaatctataagtagtaaatcagaaagtaacaatctatggcttatttctatgacaaattcaagagctagaaaaataaaatgtttcattatgcacttttagaaatgcatatttgccacaaaacctgtattactgaataatatcaaataaaatatcataaagcattttaaatatggtcttttgctgcctcatttgcaaatataatttcatataaacaatt

SEQ ID NO: 26 - DNA, 3078 bp, Homo sapiens
  misc_feature (272)..(272): Nucleotide variant
atggcccctgtgctcagtaaggactcggcggacatcgagagtatcctggctttaaatcctcgaacacaaactcatgcaactctgtgttccacttcggccaagaaattagacaagaaacattggaaaagaaatcctgataagaactgctttaattgtgagaagctggagaataattttgatgacatcaagcacacgactcttggtgagcgaggagctctccgagaagcaatgagatgcctgaaatgtgcagatgccccgtgtcagaagagctttccaactaatcttgatattaaatcattcatcacaagtattgcaaacaagaactattatggagctgctaagatgatattttctgacaacccacttggtctgacttgtggaatggtatgtccaacctctgatctttgtgtaggtggatgcaatttatatgccactgaagagggacccattaatattggtggattgcagcaatttgctactgaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaagtacttctgaaattcctcagttccggctgccgtatgatgtagtgaattttgagattgagctaatgaaggaccttggtgtaaagataatttgcggtaaaagcctttcagtgaatgaaatgactcttagcactttgaaagaaaaaggctacaaagctgctttcattggaataggtttgccagaacccaataaagatgccatcttccaaggcctgacgcaggaccaggggttttatacatccaaagactttttgccacttgtagccaaaggcagtaaagcaggaatgtgcgcctgtcactctccattgccatcgatacggggagtcgtgattgtacttggagctggagacactgcctttgactgtgcaacatctgctctacgttgtggagctcgccgtgtgttcatcgtcttcagaaaaggctttgttaatataagagctgtccctgaggagatggaacttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcgtcagtgcctttggttcagttctgagtgatcctacagtaaaagaagtcttgagccttataaaatttaacagatggggtctcccagaagtagatccagaaactatgcaaactagtgaagcatgggtatttgcaggtggtgatgtcattggtttggctaacactacagtggaatcggtgaatgatggaaagcaagcttcttggtacattcacaaatacgtacagtcacaatatggagcttccgtttctgccgagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggacattgcgacaaatgtttcccccagaatcatccggggaaccacctctggccccatgtatggccctggacaaagctcctttctgaatattgagctcatcagtgagaaaacggctgcatattggtatcaaagtgtcactgaactaaaggctgactttccagacaacattgtgattgctagcattatgtgcagttacaataaaaatgactggacggaacttgccaagaagtctgaggattctggagcagatgccctggagttaaatttatcatgtccacatggcatgggagaaagaggaatgggcctggcctgtgggcaggatccagagctgttgcggaacatctgccgctgggttaggcaagctgttcagattcctttttttgccaagctgaccccaaatgtcactgatattgtgagcatcgcaagagctgcaaaggaaggtggtgccaatggcgttacagccaccaacactgtctcaggtctgatgggattaaaatctgatggcacaccttggccagcagtggggattgcaaagcgaactacatatggaggagtgtctgggacagcaatcagacctattgctttgagagctgtgacctccattgctcgtgctctgcctggatttcccattttggctactggtggaattgactctgctgaaagtggtcttcagtttctccatagtggtgcttccgtcctccaggtatgcagtgccattcagaatcaggatttcactgtgatcaaagactactgcactggcctcaaagccctgctttatctgaaaagcattgaagaactacaagactgggatggacagagtccagctactgtgagtcaccagaaagggaaaccagttccacgtatagctgaactcatggacaagaaactgccaagttttggaccttatctggaacagcgcaagaaaatcatagcagaaaacaagattagactgaaagaacaaaatgtagctttttcaccacttaagagaaactgttttatccccaaaaggcctattcctaccaacaaggatgtaataggaaaagcactgcagtaccttggaacatttggtgaattgagcaacgtagagcaagttgtggctatgattgatgaagaaatgtgtatcaactgtggtaaacgctacatgacctgtaatgattctggctaccaggctatacagtttgatccagaaacccacctgcccaccataatcgacacttgtacaggctgtactctgtgtctcagtgtttgccctattgtcgactgcatcaaaatggtttccaggacaacaccttatgaaccaaagagaggcgtacccttatctgtgaatccggtgtgttaa

SEQ ID NO: 27 - protein, 1025 aa, Homo sapiens
  MISC_FEATURE (91)..(91): Amino acid variant
MAPVLSKDSADIESILALNPRTQTHATLCSTSAKKLDKKHWKRNPDKNCFNCEKLENNFDDIKHTTLGERGALREAMRCLKCADAPCQKSCPTNLDIKSFITSIANKNYYGAAKMIFSDNPLGLTCGMVCPTSDLCVGGCNLYATEEGPINIGGLQQFATEVFKAMSIPQIRNPSLPPPEKMSEAYSAKIALFGAGPASISCASFLARLGYSDITIFEKQEYVGGLSTSEIPQFRLPYDVVNFEIELMKDLGVKIICGKSLSVNEMTLSTLKEKGYKAAFIGIGLPEPNKDAIFQGLTQDQGFYTSKDFLPLVAKGSKAGMCACHSPLPSIRGVVIVLGAGDTAFDCATSALRCGARRVFIVFRKGFVNIRAVPEEMELAKEEKCEFLPFLSPRKVIVKGGRIVAMQFVRTEQDETGKWNEDEDQMVHLKADVVISAFGSVLSDPKVKEALSPIKFNRWGLPEVDPETMQTSEAWVFAGGDVVGLANTTVESVNDGKQASWYIHKYVQSQYGASVSAKPELPLFYTPIDLVDISVEMAGLKFINPFGLASATPATSTSMIRRAFEAGWGFALTKTFSLDKDIVTNVSPRIIRGTTSGPMYGPGQSSFLNIELISEKTAAYWCQSVTELKADFPDNIVIASIMCSYNKNDWTELAKKSEDSGADALELNLSCPHGMGERGMGLACGQDPELVRNICRWVRQAVQIPFFAKLTPNVTDIVSIARAAKEGGANGVTATNTVSGLMGLKSDGTPWPAVGIAKRTTYGGVSGTAIRPIALRAVTSIARALPGFPILATGGIDSAESGLQFLHSGASVLQVCSAIQNQDFTVIEDYCTGLKALLYLKSIEELQDWDGQSPATVSHQKGKPVPRIAELMDKKLPSFGPYLEQRKKIIAENKIRLKEQNVAFSPLKRNCFIPKRPIPTIKDVIGKALQYLGTFGELSNVEQVVAMIDEEMCINCGKCYMTCNDSGYQAIQFDPETHLPTITDTCTGCTLCLSVCPIVDCIKMVSRTTPYEPKRGVPLSVNPVC

SEQ ID NO: 28 - DNA, 99 bp, Homo sapiens
gaagcaatgagatgcctgaaatgtgcagatgccccgtgtcagaagagctttccaactaatcttgatattaaatcattcatcacaagtattgcaaacaag

SEQ ID NO: 29 - DNA, 99 bp, Homo sapiens
atggaatgaagatgaagatcagatggtccatctgaaagccgatgtggtcgtcagtgcctttggttcagttctgagtgatcctaaagtaaaagaagcctt

SEQ ID NO: 30 - DNA, 99 bp, Homo sapiens
aaagccgatgtggtcatcagtgcctttggttcagttctgagtgatcctacagtaaaagaagccttgagccctataaaatttaacagatggggtctccca

SEQ ID NO: 31 - DNA, 99 bp, Homo sapiens
gtcatcagtgcctttggttcagttctgagtgatcctaaagtaaaagaagtcttgagccctataaaatttaacagatggggtctcccagaagtagatcca

SEQ ID NO: 32 - DNA, 99 bp, Homo sapiens
gcctttggttcagttctgagtgatcctaaagtaaaagaagccttgagccttataaaatttaacagatggggtctcccagaagtagatccagaaactatg

SEQ ID NO: 33 - DNA, 99 bp, Homo sapiens
agaaactatgcaaactagtgaagcatgggtatttgcaggtggtgatgtcattggtttggctaacactacagtggaatcggtgaatgatggaaagcaagc

SEQ ID NO: 34 - DNA, 99 bp, Homo sapiens
gtacattcacaaatacgtacagtcacaatatggagcttccgtttctgccgagcctgaactacccctcttttacactcctattgatctggtggacattag

SEQ ID NO: 35 - DNA, 99 bp, Homo sapiens
ggatggggttttgccctcaccaaaactttctctcttgataaggacattgcgacaaatgtttcccccagaatcatccggggaaccacctctggccccatg

SEQ ID NO: 36 - DNA, 99 bp, Homo sapiens
tcctttctgaatattgagctcatcagtgagaaaacggctgcatattggtatcaaagtgtcactgaactaaaggctgactttccagacaacattgtgatt

SEQ ID NO: 37 - DNA, 99 bp, Homo sapiens
catgggagaaagaggaatgggcctggcctgtgggcaggatccagagctgttgcggaacatctgccgctgggttaggcaagctgttcagattcctttttt

SEQ ID NO: 38 - DNA, 99 bp, Homo sapiens
cgtcctccaggtatgcagtgccattcagaatcaggatttcactgtgatcaaagactactgcactggcctcaaagccctgctttatctgaaaagcattga

SEQ ID NO: 39 - DNA, 99 bp, Homo sapiens
tcaccacttaagagaaactgttttatccccaaaaggcctattcctaccaacaaggatgtaataggaaaagcactgcagtaccttggaacatttggtgaa

SEQ ID NO: 40 - DNA, 99 bp, Homo sapiens
gcaagttgtggctatgattgatgaagaaatgtgtatcaactgtggtaaacgctacatgacctgtaatgattctggctaccaggctatacagtttgatcc

SEQ ID NO: 41 - DNA, 99 bp, Homo sapiens
ggctaccaggctatacagtttgatccagaaacccacctgcccaccataatcgacacttgtacaggctgtactctgtgtctcagtgtttgccctattgtc

SEQ ID NO: 42 - DNA, 98 bp, Homo sapiens
gaagaactacaagactgggatggacagagtccagctactgtgagtcaccgaaagggaaaccagttccacgtatagctgaactcatggacaagaaactg

SEQ ID NO: 43 - DNA, 79 bp, Homo sapiens
  misc_feature (40)..(40): Nucleotide variant
gctagtttcaaaattttaaccatgacaattgatttccccgtaggtattcaaagcaatgagtatcccacagatcagaaat

SEQ ID NO: 44 - DNA, 79 bp, Homo sapiens
  misc_feature (40)..(40): Nucleotide variant
ggtcatttttctactgatgcctgtttttctttctcctttcttttcagtacttctgaaattcctcagttccggctgccgt

SEQ ID NO: 45 - DNA, 79 bp, Homo sapiens
  misc_feature (40)..(40): Nucleotide variant
catatgccaaattttcttatgcaaaatattgtttcttatagataatttgcggtaaaagcctttcagtgaatgaaatgac

SEQ ID NO: 46 - DNA, 79 bp, Homo sapiens
  misc_feature (40)..(40): Nucleotide variant
aatggtttctctacttgaaaatggggttttcctttcatcattcaggaatgtgcgcctgtcactctccattgccatcgat

SEQ ID NO: 47 - DNA, 79 bp, Homo sapiens
  misc_feature (40)..(40): Nucleotide variant
tttgtggtcagtgacatcaataccctctatttctgtttgtaggctatacagtttgatccagaaacccacctgcccacca

SEQ ID NO: 48 - protein, 51 aa, Homo sapiens
TLGERGALREAMRCLKCADAPCQKSFPTNLDIKSFITSIANKNYYGAAKMI

SEQ ID NO: 49 - protein, 51 aa, Homo sapiens
RTEQDETGKWNEDEDQMVHLKADVVVSAFGSVLSDPKVKEALSPIKFNRWG

SEQ ID NO: 50 - protein, 51 aa, Homo sapiens
EDEDQMVHLKADVVISAFGSVLSDPTVKEALSPIKFNRWGLPEVDPETMQT

SEQ ID NO: 51 - protein, 51 aa, Homo sapiens
QMVHLKADVVISAFGSVLSDPKVKEVLSPIKFNRWGLPEVDPETMQTSEAW

SEQ ID NO: 52 - protein, 51 aa, Homo sapiens
HLKADVVISAFGSVLSDPKVKEALSLIKFNRWGLPEVDPETMQTSEAWVFA

SEQ ID NO: 53 - protein, 51 aa, Homo sapiens
RWGLPEVDPETMQTSEAWVFAGGDVIGLANTTVESVNDGKQASWYIHKYVQ

SEQ ID NO: 54 - protein, 51 aa, Homo sapiens
VNDGKQASWYIHKYVQSQYGASVSAEPELPLFYTPIDLVDISVEMAGLKFI

SEQ ID NO: 55 - protein, 51 aa, Homo sapiens
SMIRRAFEAGWGFALTKTFSLDKDIATNVSPRIIRGTTSGPMYGPGQSSFL

SEQ ID NO: 56 - protein, 51 aa, Homo sapiens
GPMYGPGQSSFLNIELISEKTAAYWYQSVTELKADFPDNIVIASIMCSYNK

SEQ ID NO: 57 - protein, 51 aa, Homo sapiens
ELNLSCPHGMGERGMGLACGQDPELLRNICRWVRQAVQIPFFAKLTPNVTD

SEQ ID NO: 58 - protein, 51 aa, Homo sapiens
LQFLHSGASVLQVCSAIQNQDFTVIKDYCTGLKALLYLKSIEELQDWDGQS

SEQ ID NO: 59 - protein, 51 aa, Homo sapiens
RLKEQNVAFSPLKRNCFIPKRPIPTNKDVIGKALQYLGTFGELSNVEQVVA

SEQ ID NO: 60 - protein, 51 aa, Homo sapiens
TFGELSNVEQVVAMIDEEMCINCGKRYMTCNDSGYQAIQFDPETHLPTITD

SEQ ID NO: 61 - protein, 51 aa, Homo sapiens
KCYMTCNDSGYQAIQFDPETHLPTIIDTCTGCTLCLSVCPIVDCIKMVSRT

SEQ ID NO: 62 - protein, 867 aa, Homo sapiens
MAPVLSKDSADIESILALNPRTQTHATLCSTSAKKLDKKHWKRNPDKNCFNCEKLENNFDDIKHTTLGERGALREAMRCLKCADAPCQKSFPTNLDIKSFITSIANKNYYGAAKMIFSDNPLGLTCGMVCPTSDLCVGGCNLYATEEGPINIGGLQQFATEVFKAMSIPQIRNPSLPPPEKMSEAYSAKIALFGAGPASISCASFLARLGYSDITIFEKQEYVGGLSTSEIPQFRLPYDVVNFEIELMKDLGVKIICGKSLSVNEMTLSTLKEKGYKAAFIGIGLPEPNKDAIFQGLTQDQGFYTSKDFLPLVAKGSKAGMCACHSPLPSIRGVVIVLGAGDTAFDCATSALRCGARRVFIVFRKGFVNIRAVPEEMELAKEEKCEFLPFLSPRKVIVKGGRIVAMQFVRTEQDETGKWNEDEDQMVHLKADVVVSAFGSVLSDPTVKEVLSLIKFNRWGLPEVDPETMQTSEAWVFAGGDVIGLANTTVESVNDGKQASWYIHKYVQSQYGASVSAEPELPLFYTPIDLVDISVEMAGLKFINPFGLASATPATSTSMIRRAFEAGWGFALTKTFSLDKDIATNVSPRIIRGTTSGPMYGPGQSSFLNIELISEKTAAYWYQSVTELKADFPDNIVIASIMCSYNKNDWTELAKKSEDSGADALELNLSCPHGMGERGMGLACGQDPELLRNICRWVRQAVQIPFFAKLTPNVTDIVSIARAAKEGGANGVTATNTVSGLMGLKSDGTPWPAVGIAKRTTYGGVSGTAIRPIALRAVTSIARALPGFPILATGGIDSAESGLQFLHSGASVLQVCSAIQNQDFTVIKDYCTGLKALLYLKSIEELQDWDGQSPATVSHRKGNQFHV

SEQ ID NO: 63 - protein, 49 aa, Homo sapiens
IQNQDFTVIEDYCTGLKALLYLKSIEELQDWDGQSPATVSHRKGNQFHV

SEQ ID NO: 64 - DNA, 49 bp, Homo sapiens
cagatgccccgtgtcagaagagctttccaactaatcttgatattaaatc

SEQ ID NO: 65 - DNA, 49 bp, Homo sapiens
gtccatctgaaagccgatgtggtcgtcagtgcctttggttcagttctga

SEQ ID NO: 66 - DNA, 49 bp, Homo sapiens
ttggttcagttctgagtgatcctacagtaaaagaagccttgagccctat

SEQ ID NO: 67 - DNA, 49 bp, Homo sapiens
tgagtgatcctaaagtaaaagaagtcttgagccctataaaatttaacag

SEQ ID NO: 68 - DNA, 49 bp, Homo sapiens
ctaaagtaaaagaagccttgagccttataaaatttaacagatggggtct

SEQ ID NO: 69 - DNA, 49 bp, Homo sapiens
tgggtatttgcaggtggtgatgtcattggtttggctaacactacagtgg

SEQ ID NO: 70 - DNA, 49 bp, Homo sapiens
caatatggagcttccgtttctgccgagcctgaactacccctcttttaca

SEQ ID NO: 71 - DNA, 49 bp, Homo sapiens
ctttctctcttgataaggacattgcgacaaatgtttcccccagaatcat

SEQ ID NO: 72 - DNA, 49 bp, Homo sapiens
gtgagaaaacggctgcatattggtatcaaagtgtcactgaactaaaggc

SEQ ID NO: 73 - DNA, 49 bp, Homo sapiens
gcctgtgggcaggatccagagctgttgcggaacatctgccgctgggtta

SEQ ID NO: 74 - DNA, 49 bp, Homo sapiens
cagaatcaggatttcactgtgatcaaagactactgcactggcctcaaag

SEQ ID NO: 75 - DNA, 49 bp, Homo sapiens
tccccaaaaggcctattcctaccaacaaggatgtaataggaaaagcact

SEQ ID NO: 76 - DNA, 49 bp, Homo sapiens
gaaatgtgtatcaactgtggtaaacgctacatgacctgtaatgattctg

SEQ ID NO: 77 - DNA, 49 bp, Homo sapiens
cagaaacccacctgcccaccataatcgacacttgtacaggctgtactct

SEQ ID NO: 78 - DNA, 48 bp, Homo sapiens
agagtccagctactgtgagtcaccgaaagggaaaccagttccacgtat

SEQ ID NO: 79 - DNA, 49 bp, Homo sapiens
ttaaccatgacaattgatttccccataggtattcaaagcaatgagtatc

SEQ ID NO: 80 - DNA, 49 bp, Homo sapiens
gatgcctgtttttctttctccttttttttcagtacttctgaaattcctc

SEQ ID NO: 81 - DNA, 49 bp, Homo sapiens
cttatgcaaaatattgtttcttatggataatttgcggtaaaagcctttc

SEQ ID NO: 82 - DNA, 49 bp, Homo sapiens
tgaaaatggggttttcctttcatcgttcaggaatgtgcgcctgtcactc

SEQ ID NO: 83 - DNA, 49 bp, Homo sapiens
atcaataccctctatttctgtttgtaggctatacagtttgatccagaaa

SEQ ID NO: 84 - protein, 49 aa, Homo sapiens
LGERGALREAMRCLKCADAPCQKSFPTNLDIKSFITSIANKNYYGAAKM

SEQ ID NO: 85 - protein, 49 aa, Homo sapiens
TEQDETGKWNEDEDQMVHLKADVVVSAFGSVLSDPKVKEALSPIKFNRW

SEQ ID NO: 86 - protein, 49 aa, Homo sapiens
DEDQMVHLKADVVISAFGSVLSDPTVKEALSPIKFNRWGLPEVDPETMQ

SEQ ID NO: 87 - protein, 49 aa, Homo sapiens
MVHLKADVVISAFGSVLSDPKVKEVLSPIKFNRWGLPEVDPETMQTSEA

SEQ ID NO: 88 - protein, 49 aa, Homo sapiens
LKADVVISAFGSVLSDPKVKEALSLIKFNRWGLPEVDPETMQTSEAWVF

SEQ ID NO: 89 - protein, 49 aa, Homo sapiens
WGLPEVDPETMQTSEAWVFAGGDVIGLANTTVESVNDGKQASWYIHKYV

SEQ ID NO: 90 - protein, 49 aa, Homo sapiens
NDGKQASWYIHKYVQSQYGASVSAEPELPLFYTPIDLVDISVEMAGLKF

SEQ ID NO: 91 - protein, 49 aa, Homo sapiens
MIRRAFEAGWGFALTKTFSLDKDIATNVSPRIIRGTTSGPMYGPGQSSF

SEQ ID NO: 92 - protein, 49 aa, Homo sapiens
PMYGPGQSSFLNIELISEKTAAYWYQSVTELKADFPDNIVIASIMCSYN

SEQ ID NO: 93 - protein, 49 aa, Homo sapiens
LNLSCPHGMGERGMGLACGQDPELLRNICRWVRQAVQIPFFAKLTPNVT

SEQ ID NO: 94 - protein, 49 aa, Homo sapiens
QFLHSGASVLQVCSAIQNQDFTVIKDYCTGLKALLYLKSIEELQDWDGQ

SEQ ID NO: 95 - protein, 49 aa, Homo sapiens
LKEQNVAFSPLKRNCFIPKRPIPTNKDVIGKALQYLGTFGELSNVEQVV

SEQ ID NO: 96 - protein, 49 aa, Homo sapiens
FGELSNVEQVVAMIDEEMCINCGKRYMTCNDSGYQAIQFDPETHLPTIT

SEQ ID NO: 97 - protein, 49 aa, Homo sapiens
CYMTCNDSGYQAIQFDPETHLPTIIDTCTGCTLCLSVCPIVDCIKMVSR

SEQ ID NO: 98 - DNA, 25 bp, Homo sapiens
ttccaactaatcttgatattaaatc

SEQ ID NO: 99 - DNA, 25 bp, Homo sapiens
gtccatctgaaagccgatgtggtcg

SEQ ID NO: 100 - DNA, 4451 bp, Homo sapiens
gctccgcccccgcgccgccggccctagtctgcctgttttcgactcgcgctccggctgctgtcacttggctctctggctggagcttgaggacgcaaggagggtttgtcactggcagactcgagactgtaggcactgccatggcccctgtgctcagtaaggactcggcggacatcgagagtatcctggctttaaatcctcgaacacaaactcatgcaactctgtgttccacttcggccaagaaattagacaagaaacattggaaaagaaatcctgataagaactgctttaattgtgagaagctggagaataattttgatgacatcaagcacacgactcttggtgagcgaggagctctccgagaagcaatgagatgcctgaaatgtgcagatgccccgtgtcagaagagctgtccaactaatcttgatattaaatcattcatcacaagtattgcaaacaagaactattatggagctgctaagatgatattttctgacaacccacttggtctgacttgtggaatggtatgtccaacctctgatctttgtgtaggtggatgcaatttatatgccactgaagagggacccattaatattggtggattgcagcaatttgctactgaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaagtacttctgaaattcctcagttccggctgccgtatgatgtagtgaattttgagattgagctaatgaaggaccttggtgtaaagataatttgcggtaaaagcctttcagtgaatgaaatgactcttagcactttgaaagaaaaaggctacaaagctgctttcattggaataggtttgccagaacccaataaagatgccatcttccaaggcctgacgcaggaccaggggttttatacatccaaagactttttgccacttgtagccaaaggcagtaaagcaggaatgtgcgcctgtcactctccattgccatcgatacggggagtcgtgattgtacttggagctggagacactgcctttgactgtgcaacatctgctctacgttgtggagctcgccgtgtgttcatcgtcttcagaaaaggctttgttaatataagagctgtccctgaggagatggaacttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcatcagtgcctttggttcagttctgagtgatcctaaagtaaaagaagccttgagccctataaaatttaacagatggggtctcccagaagtagatccagaaactatgcaaactagtgaagcatgggtatttgcaggtggtgatgtcgttggtttggctaacactacagtggaatcggtgaatgatggaaagcaagcttcttggtacattcacaaatacgtacagtcacaatatggagcttccgtttctgccaagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggacattgtgacaaatgtttcccccagaatcatccggggaaccacctctggccccatgtatggccctggacaaagctcctttctgaatattgagctcatcagtgagaaaacggctgcatattggtgtcaaagtgtcactgaactaaaggctgactttccagacaacattgtgattgctagcattatgtgcagttacaataaaaatgactggacggaacttgccaagaagtctgaggattctggagcagatgccctggagttaaatttatcatgtccacatggcatgggagaaagaggaatgggcctggcctgtgggcaggatccagagctggtgcggaacatctgccgctgggttaggcaagctgttcagattcctttttttgccaagctgaccccaaatgtcactgatattgtgagcatcgcaagagctgcaaaggaaggtggtgccaatggcgttacagccaccaacactgtctcaggtctgatgggattaaaatctgatggcacaccttggccagcagtggggattgcaaagcgaactacatatggaggagtgtctgggacagcaatcagacctattgctttgagagctgtgacctccattgctcgtgctctgcctggatttcccattttggctactggtggaattgactctgctgaaagtggtcttcagtttctccatagtggtgcttccgtcctccaggtatgcagtgccattcagaatcaggatttcactgtgatcgaagactactgcactggcctcaaagccctgctttatctgaaaagcattgaagaactacaagactgggatggacagagtccagctactgtgagtcaccagaaagggaaaccagttccacgtatagctgaactcatggacaagaaactgccaagttttggaccttatctggaacagcgcaagaaaatcatagcagaaaacaagattagactgaaagaacaaaatgtagctttttcaccacttaagagaaactgttttatccccaaaaggcctattcctaccatcaaggatgtaataggaaaagcactgcagtaccttggaacatttggtgaattgagcaacgtagagcaagttgtggctatgattgatgaagaaatgtgtatcaactgtggtaaatgctacatgacctgtaatgattctggctaccaggctatacagtttgatccagaaacccacctgcccaccataaccgacacttgtacaggctgtactctgtgtctcagtgtttgccctattgtcgactgcatcaaaatggtttccaggacaacaccttatgaaccaaagagaggcgtacccttatctgtgaatccggtgtgttaaggtgatttgtgaaacagttgctgtgaactttcatgtcacctacatatgctgatcttttaaaatcatgatccttgtgttcagctctttccaaattaaaacaaatatacattttctaaataaaaatatgtaatttcaaaatacatttgtaagtgtaaaaaatgtctcatgtcaatgaccattcaattagtggtcataaaatagaataattcttttctgaggatagtagttaaataactgtgtggcagttaattggatgttcactgccagttgtcttatgtgaaaaattaacttttttgtggcaattagtgtgacagtttccaaattgccctatgctgtgctccatatttgatttctaattgtaagtgaaattaagcattttgaaacaaagtactctttaacatacaagaaaatgtatccaaggaaacattttatcattaaaaattacctttaattttaatgctgtttctaagaaaatgtagttagctccataaagtacaaatgaagaaagtcaaaaaattatttgctatggcaggataagaaagcctaaaattgagtttgtagaactttattaagtaaaatccccttcgctgaaattgcttatttttggtgttggatagaggatagggagaatatttactaactaaataccattcactactcatgcgtgagatgggtgtacaaactcatcctcttttaatggcatttctctttaaactatgttcctaacaaaatgagatgataggatagatcctggttaccactcttttgctgtgcacatacgggctctgactggttttaatagtcaccttcatgattatagcaactaatgtttgaacaaagctcaaagtatgcaatgcttcattattcaagaatgaaaaatataatgttgataatatatattaagtgtgccaaatcagtttgactactctctgttttagtgtttatgtttaaaagaaatatattttttgttattattagataatatttttgtatttctctattttcataatcagtaaatagtgtcatataaactcatttatctcctcttcatggcatcttcaatatgaatctataagtagtaaatcagaaagtaacaatctatggcttatttctatgacaaattcaagagctagaaaaataaaatgtttcattatgcacttttagaaatgcatatttgccacaaaacctgtattactgaataatatcaaataaaatatcataaagcattttaaaaaa

SEQ ID NO: 101 - DNA, 3078 bp, Homo sapiens
  misc_feature (272)..(272): Nucleotide variant
atggcccctgtgctcagtaaggactcggcggacatcgagagtatcctggctttaaatcctcgaacacaaactcatgcaactctgtgttccacttcggccaagaaattagacaagaaacattggaaaagaaatcctgataagaactgctttaattgtgagaagctggagaataattttgatgacatcaagcacacgactcttggtgagcgaggagctctccgagaagcaatgagatgcctgaaatgtgcagatgccccgtgtcagaagagctgtccaactaatcttgatattaaatcattcatcacaagtattgcaaacaagaactattatggagctgctaagatgatattttctgacaacccacttggtctgacttgtggaatggtatgtccaacctctgatctttgtgtaggtggatgcaatttatatgccactgaagagggacccattaatattggtggattgcagcaatttgctactgaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaagtacttctgaaattcctcagttccggctgccgtatgatgtagtgaattttgagattgagctaatgaaggaccttggtgtaaagataatttgcggtaaaagcctttcagtgaatgaaatgactcttagcactttgaaagaaaaaggctacaaagctgctttcattggaataggtttgccagaacccaataaagatgccatcttccaaggcctgacgcaggaccaggggttttatacatccaaagactttttgccacttgtagccaaaggcagtaaagcaggaatgtgcgcctgtcactctccattgccatcgatacggggagtcgtgattgtacttggagctggagacactgcctttgactgtgcaacatctgctctacgttgtggagctcgccgtgtgttcatcgtcttcagaaaaggctttgttaatataagagctgtccctgaggagatggaacttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcatcagtgcctttggttcagttctgagtgatcctaaagtaaaagaagccttgagccctataaaatttaacagatggggtctcccagaagtagatccagaaactatgcaaactagtgaagcatgggtatttgcaggtggtgatgtcgttggtttggctaacactacagtggaatcggtgaatgatggaaagcaagcttcttggtacattcacaaatacgtacagtcacaatatggagcttccgtttctgccaagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggacattgtgacaaatgtttcccccagaatcatccggggaaccacctctggccccatgtatggccctggacaaagctcctttctgaatattgagctcatcagtgagaaaacggctgcatattggtgtcaaagtgtcactgaactaaaggctgactttccagacaacattgtgattgctagcattatgtgcagttacaataaaaatgactggacggaacttgccaagaagtctgaggattctggagcagatgccctggagttaaatttatcatgtccacatggcatgggagaaagaggaatgggcctggcctgtgggcaggatccagagctggtgcggaacatctgccgctgggttaggcaagctgttcagattcctttttttgccaagctgaccccaaatgtcactgatattgtgagcatcgcaagagctgcaaaggaaggtggtgccaatggcgttacagccaccaacactgtctcaggtctgatgggattaaaatctgatggcacaccttggccagcagtggggattgcaaagcgaactacatatggaggagtgtctgggacagcaatcagacctattgctttgagagctgtgacctccattgctcgtgctctgcctggatttcccattttggctactggtggaattgactctgctgaaagtggtcttcagtttctccatagtggtgcttccgtcctccaggtatgcagtgccattcagaatcaggatttcactgtgatcgaagactactgcactggcctcaaagccctgctttatctgaaaagcattgaagaactacaagactgggatggacagagtccagctactgtgagtcaccagaaagggaaaccagttccacgtatagctgaactcatggacaagaaactgccaagttttggaccttatctggaacagcgcaagaaaatcatagcagaaaacaagattagactgaaagaacaaaatgtagctttttcaccacttaagagaaactgttttatccccaaaaggcctattcctaccatcaaggatgtaataggaaaagcactgcagtaccttggaacatttggtgaattgagcaacgtagagcaagttgtggctatgattgatgaagaaatgtgtatcaactgtggtaaatgctacatgacctgtaatgattctggctaccaggctatacagtttgatccagaaacccacctgcccaccataaccgacacttgtacaggctgtactctgtgtctcagtgtttgccctattgtcgactgcatcaaaatggtttccaggacaacaccttatgaaccaaagagaggcgtacccttatctgtgaatccggtgtgttaa

SEQ ID NO: 102 - DNA, 297 bp, Homo sapiens
  variation (47)..(47)
  variant (47)..(47)
gatgtaagctagtttcaaaattttaaccatgacaattgatttccccataggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaaggtaatgcctacatttatttcatgtttatagtgtcagaaatgatggagcaa

SEQ ID NO: 103 - DNA, 188 bp, Homo sapiens
  variant (49)..(49)
ttaattaatcatatgccaaattttcttatgcaaaatattgtttcttatggataatttgcggtaaaagcctttcagtgaatgaaatgactcttagcactttgaaagaaaaaggctacaaagctgctttcattggaataggtgagtagtttactcttaaatatatttatttataatgtatttttttattt

SEQ ID NO: 104 - DNA, 1503 bp, Homo sapiens
  variant (48)..(48)
tgttgacctttgtggtcagtgacatcaataccctctatttctgtttgtaggctatacagtttgatccagaaacccacctgcccaccataaccgacacttgtacaggctgtactctgtgtctcagtgtttgccctattgtcgactgcatcaaaatggtttccaggacaacaccttatgaaccaaagagaggcgtacccttatctgtgaatccggtgtgttaaggtgatttgtgaaacagttgctgtgaactttcatgtcacctacatatgctgatcttttaaaatcatgatccttgtgttcagctctttccaaattaaaacaaatatacattttctaaataaaaatatgtaatttcaaaatacatttgtaagtgtaaaaaatgtctcatgtcaatgaccattcaattagtggtcataaaatagaataattcttttctgaggatagtagttaaataactgtgtggcagttaattggatgttcactgccagttgtcttatgtgaaaaattaacttttttgtggcaattagtgtgacagtttccaaattgccctatgctgtgctccatatttgatttctaattgtaagtgaaattaagcattttgaaacaaagtactctttaacatacaagaaaatgtatccaaggaaacattttatcattaaaaattacctttaattttaatgctgtttctaagaaaatgtagttagctccataaagtacaaatgaagaaagtcaaaaaattatttgctatggcaggataagaaagcctaaaattgagtttgtagaactttattaagtaaaatccccttcgctgaaattgcttatttttggtgttggatagaggatagggagaatatttactaactaaataccattcactactcatgcgtgagatgggtgtacaaactcatcctcttttaatggcatttctctttaaactatgttcctaacaaaatgagatgataggatagatcctggttaccactcttttgctgtgcacatacgggctctgactggttttaatagtcaccttcatgattatagcaactaatgtttgaacaaagctcaaagtatgcaatgcttcattattcaagaatgaaaaatataatgttgataatatatattaagtgtgccaaatcagtttgactactctctgttttagtgtttatgtttaaaagaaatatattttttgttattattagataatatttttgtatttctctattttcataatcagtaaatagtgtcatataaactcatttatctcctcttcatggcatcttcaatatgaatctataagtagtaaatcagaaagtaacaatctatggcttatttctatgacaaattcaagagctagaaaaataaaatgtttcattatgcacttttagaaatgcatatttgccacaaaacctgtattactgaataatatcaaataaaatatcataaagcattttaaatatggtcttttgctgcctcatttgcaaatataatttcatataaacaatt